SEQUENCE LISTING (87 sequences)

SEQ ID NO: 1 - RNA, 34 nt, Homo sapiens
ggcucguuggucuagggguaugauucucgcuuag

SEQ ID NO: 2 - RNA, 33 nt, Homo sapiens
ggggguauagcucagggguagagcauuugacug

SEQ ID NO: 3 - RNA, 33 nt, Homo sapiens
gggcguauagcucagggguagagcauuugacug

SEQ ID NO: 4 - RNA, 33 nt, Homo sapiens
ggggauauagcucagggguagagcauuugacug

SEQ ID NO: 5 - RNA, 33 nt, Homo sapiens
ggggguauagcucagggguagagcacuugacug

SEQ ID NO: 6 - RNA, 33 nt, Homo sapiens
ggggguauagcucagugguagagcauuugacug

SEQ ID NO: 7 - RNA, 33 nt, Homo sapiens
ggggguauagcuuagggguagagcauuugacug

SEQ ID NO: 8 - RNA, 33 nt, Homo sapiens
ggggguauaguucagggguagagcauuugacug

SEQ ID NO: 9 - RNA, 34 nt, Homo sapiens
ggggguauagcucaggugguagagcauuugacug

SEQ ID NO: 10 - RNA, 34 nt, Homo sapiens
ggggguauagcucaguggguagagcauuugacug

SEQ ID NO: 11 - RNA, 33 nt, Homo sapiens
gggggcauagcucagugguagagcauuugacug

SEQ ID NO: 12 - RNA, 33 nt, Homo sapiens
ggggguauagcucacagguagagcauuugacug

SEQ ID NO: 13 - RNA, 33 nt, Homo sapiens
ggggguauagcuuagcgguagagcauuugacug

SEQ ID NO: 14 - RNA, 33 nt, Homo sapiens
ggggguguagcucagugguagagcauuugacug

SEQ ID NO: 15 - RNA, 34 nt, Homo sapiens
ggggguauaucucagggggcagagcauuugacug

SEQ ID NO: 16 - RNA, 33 nt, Homo sapiens
gcucguuggucuagggguaugauucucgcuucg

SEQ ID NO: 17 - RNA, 32 nt, Homo sapiens
cucguuggucuagggguaugauucucgcuucg

SEQ ID NO: 18 - RNA, 28 nt, Homo sapiens
uuggucuagggguaugauucucgcuucg

SEQ ID NO: 19 - RNA, 30 nt, Homo sapiens
cguuggucuagggguaugauucucgcuucg

SEQ ID NO: 20 - RNA, 29 nt, Homo sapiens
guuggucuagggguaugauucucgcuucg

SEQ ID NO: 21 - RNA, 33 nt, Homo sapiens
ggcucguuggucuagggguaugauucucgcuuc

SEQ ID NO: 22 - RNA, 32 nt, Homo sapiens
ggcucguuggucuagggguaugauucucgcuu

SEQ ID NO: 23 - RNA, 31 nt, Homo sapiens
ggcucguuggucuagggguaugauucucgcu

SEQ ID NO: 24 - RNA, 30 nt, Homo sapiens
ggcucguuggucuagggguaugauucucgc

SEQ ID NO: 25 - RNA, 29 nt, Homo sapiens
ggcucguuggucuagggguaugauucucg

SEQ ID NO: 26 - RNA, 32 nt, Homo sapiens
gcucguuggucuagggguaugauucucgcuuc

SEQ ID NO: 27 - RNA, 30 nt, Homo sapiens
cucguuggucuagggguaugauucucgcuu

SEQ ID NO: 28 - RNA, 28 nt, Homo sapiens
ucguuggucuagggguaugauucucgcu

SEQ ID NO: 29 - RNA, 26 nt, Homo sapiens
cguuggucuagggguaugauucucgc

SEQ ID NO: 30 - RNA, 24 nt, Homo sapiens
guuggucuagggguaugauucucg

SEQ ID NO: 31 - RNA, 35 nt, Homo sapiens
ggcucguuggucuagggguaugauucucgcuucgg

SEQ ID NO: 32 - RNA, 36 nt, Homo sapiens
ggcucguuggucuagggguaugauucucgcuucggg

SEQ ID NO: 33 - RNA, 37 nt, Homo sapiens
ggcucguuggucuagggguaugauucucgcuucgggu

SEQ ID NO: 34 - RNA, 32 nt, Homo sapiens
gggguauagcucagggguagagcauuugacug

SEQ ID NO: 35 - RNA, 31 nt, Homo sapiens
ggguauagcucagggguagagcauuugacug

SEQ ID NO: 36 - RNA, 30 nt, Homo sapiens
gguauagcucagggguagagcauuugacug

SEQ ID NO: 37 - RNA, 29 nt, Homo sapiens
guauagcucagggguagagcauuugacug

SEQ ID NO: 38 - RNA, 28 nt, Homo sapiens
uauagcucagggguagagcauuugacug

SEQ ID NO: 39 - RNA, 32 nt, Homo sapiens
ggggguauagcucagggguagagcauuugacu

SEQ ID NO: 40 - RNA, 31 nt, Homo sapiens
ggggguauagcucagggguagagcauuugac

SEQ ID NO: 41 - RNA, 30 nt, Homo sapiens
ggggguauagcucagggguagagcauuuga

SEQ ID NO: 42 - RNA, 29 nt, Homo sapiens
ggggguauagcucagggguagagcauuug

SEQ ID NO: 43 - RNA, 28 nt, Homo sapiens
ggggguauagcucagggguagagcauuu

SEQ ID NO: 44 - RNA, 31 nt, Homo sapiens
gggguauagcucagggguagagcauuugacu

SEQ ID NO: 45 - RNA, 29 nt, Homo sapiens
ggguauagcucagggguagagcauuugac

SEQ ID NO: 46 - RNA, 27 nt, Homo sapiens
gguauagcucagggguagagcauuuga

SEQ ID NO: 47 - RNA, 25 nt, Homo sapiens
guauagcucagggguagagcauuug

SEQ ID NO: 48 - RNA, 23 nt, Homo sapiens
uauagcucagggguagagcauuu

SEQ ID NO: 49 - RNA, 34 nt, Homo sapiens
ggggguauagcucagggguagagcauuugacugc

SEQ ID NO: 50 - RNA, 35 nt, Homo sapiens
ggggguauagcucagggguagagcauuugacugca

SEQ ID NO: 51 - RNA, 36 nt, Homo sapiens
ggggguauagcucagggguagagcauuugacugcag

SEQ ID NO: 52 - RNA, 37 nt, Homo sapiens
ggggguauagcucagggguagagcauuugacugcaga

SEQ ID NO: 53 - protein, 34 aa, Mus musculus
SVSEIQLMHNLGKHLNSMERVEWLRKKLQDVHNY

SEQ ID NO: 54 - DNA, 21 bp, Mus musculus
ggagcgggagaaatggatatg

SEQ ID NO: 55 - DNA, 21 bp, Mus musculus
aaagtcgctctgagttgttat

SEQ ID NO: 56 - DNA, 20 bp, Mus musculus
aagaccgcgaagagtttgtc

SEQ ID NO: 57 - DNA, 26 bp, Mus musculus
ctggtcgagctggacggcgacgtaac

SEQ ID NO: 58 - DNA, 23 bp, Mus musculus
attgatcgcgcttctcgttgggg

SEQ ID NO: 59 - DNA, 20 bp, Mus musculus
ctcttcatgaggaggaccct

SEQ ID NO: 60 - DNA, 21 bp, Mus musculus
gccaggcaggtgcctggacat

SEQ ID NO: 61 - DNA, 22 bp, Mus musculus
gaccaggttcgttcactcatgg

SEQ ID NO: 62 - DNA, 24 bp, Mus musculus
aggctaagtgccttctctctacac

SEQ ID NO: 63 - DNA, 22 bp, Mus musculus
gagtctacatgtctagggtcta

SEQ ID NO: 64 - DNA, 19 bp, Mus musculus
tagttggcccacgtcctgt

SEQ ID NO: 65 - DNA, 20 bp, Mus musculus
ggacgacggcaactacaaga

SEQ ID NO: 66 - DNA, 20 bp, Mus musculus
ttgtactccagcttgtgccc

SEQ ID NO: 67 - DNA, 21 bp, Mus musculus
aggtcggtgtgaacggatttg

SEQ ID NO: 68 - DNA, 23 bp, Mus musculus
tgtagaccatgtagttgaggtca

SEQ ID NO: 69 - RNA, 34 nt, Mus musculus
ggcucguuggucuagggguaugauucucgcuucg

SEQ ID NO: 70 - RNA, 33 nt, Mus musculus
gcggguauagcucagggguagaauauuugacug

SEQ ID NO: 71 - RNA, 33 nt, Mus musculus
ggggguguagcucagugguagagcgcgugcuua

SEQ ID NO: 72 - RNA, 35 nt, Mus musculus
guagucguggccgagugguuaaggcgauggacuug

SEQ ID NO: 73 - RNA, 35 nt, Mus musculus
gcauuggugguucagugguagaauucucgccugcc

SEQ ID NO: 74 - RNA, 35 nt, Mus musculus
ucccugguggucuagugguuaggauucggcgcucu

SEQ ID NO: 75 - RNA, 35 nt, Mus musculus
ucccugguggucuaguggcuaggauucggcgcuuu

SEQ ID NO: 76 - RNA, 35 nt, Mus musculus
guuuccguaguguagugguuaucacguucgccuca

SEQ ID NO: 77 - RNA, 35 nt, Mus musculus
gccgagaucguauagugguuaguacucugcauugu

SEQ ID NO: 78 - RNA, 29 nt, Mus musculus
ugugagucacgugagggcagaaucugcuc

SEQ ID NO: 79 - DNA, 32 bp, Mus musculus
aagcacgcgctctaccactgagctacaccccc

SEQ ID NO: 80 - DNA, 32 bp, Mus musculus
agtcaaatattctacccctgagctatacccgc

SEQ ID NO: 81 - DNA, 33 bp, Mus musculus
aatgcagagtactaaccactatacgatctcggc

SEQ ID NO: 82 - DNA, 32 bp, Mus musculus
aagcgagaatcatacccctagaccaacgagcc

SEQ ID NO: 83 - DNA, 33 bp, Mus musculus
agtccatcgccttaaccactcggccacgactac

SEQ ID NO: 84 - DNA, 33 bp, Mus musculus
aggcgaacgtgataaccactacactacggaaac

SEQ ID NO: 85 - DNA, 31 bp, Mus musculus
cgccgaatcctaaccactagaccaccaggga

SEQ ID NO: 86 - DNA, 33 bp, Mus musculus
agcgccgaatcctagccactagaccaccaggga

SEQ ID NO: 87 - DNA, 28 bp, Mus musculus
gagaattctaccactgaaccacccatgc

(19) United States
(12) Patent Application Publication
(10) Pub. No.: US 2024/0018517 A1
(43) Pub. Date: Jan. 18, 2024

(54) MODULATING HEMATAOPOIESIS AND MYLEOID CELL PRODUCTION

(71) Applicants: The General Hospital Corporation, Boston, MA (US); President and Fellows of Harvard College, Cambridge, MA (US)

(72) Inventors: David T. SCADDEN, Weston, MA (US); Youmna KFOURY, Cambridge, MA (US)

(21) Appl. No.: 18/036,473

(22) PCT Filed: Nov. 12, 2021
(86) PCT No.: PCT/US2021/059264

§ 371 Date: May 11, 2023

Related U.S. Application Data
(60) Provisional application No. 63/113,056, filed on Nov. 12, 2020.

(51) Int. Cl. C12N 15/113; C12N 5/0789; A61P 31/10; A61K 31/7105

(52) U.S. Cl. CPC C12N 15/113; C12N 5/0647; C12N 2320/30; A61K 31/7105; C12N 2310/344; A61P 31/10

(57) ABSTRACT

The invention features a method of treating a disease or disorder in a subject, the method comprising administering a therapeutically effective amount of a 5′-tiRNA to treat the disease or disorder in the subject.

### CROSS-REFERENCE TO RELATED APPLICATIONS

This application claims benefit of U.S. Provisional Application No. 63/113,056 filed Nov. 12, 2020, which is hereby incorporated by reference in its entirety.

### SEQUENCE LISTING

The instant application contains a Sequence Listing which has been submitted electronically in ASCII format and is hereby incorporated by reference in its entirety. The ASCII copy, created on Nov. 12, 2021, is named 51627-002WO2_Sequence_Listing_11_12_21_ST25 and is 13,392 bytes in size.

### BACKGROUND OF THE INVENTION

This invention relates to truncated tRNAs and methods of using the same for modulating gene expression, cell differentiation, and development such as during hematopoiesis as well as for treating subjects.

Stem cell niches are specialized local microenvironments that modulate stem and progenitor populations of a tissue. They have largely been defined in terms of the cells comprising them and the cytokines or adhesion molecules produced by them. There is accordingly a need in the art for developing modifying polynucleotides for improving phenotypes and genotypes and developmental pathways of such niches and the cells forming them.

### SUMMARY OF THE INVENTION

In one aspect, the invention, in general, features, a synthetic 5′-tiRNA. In some embodiments, the 5′-tiRNA is between 30-37 nucleotides and includes nucleotides capable of forming a tRNA D-arm. In other embodiments, the 5′-tiRNA is modified (e.g., the 5′-tiRNA includes a non-natural or modified nucleoside or nucleotide). Exemplary modifications are chosen from 2′-O-methyl (2′-O-Me) modified nucleoside, a phosphorothioate (PS) bond between nucleosides; and a 2′-fluoro (2′-F) modified nucleoside. In some embodiments, the 5′-tiRNA has sequence identity to 5′-ti-Pro-CGG-1-1: GGCUCGUUGGUCUAGGGGUAUGAUUCUCGCUUCG (SEQ ID NO: 1). In embodiments, the 5′-tiRNA is 5′-ti-Pro-CGG-1 (SEQ ID NO: 1). In other embodiments, the 5′-tiRNA has sequence identity to 5′-ti-Cys-GCA-10-1: GGGGGUAUAGCUCAGGGGUAGAGCAUUUGACUG (SEQ ID NO: 2). In still other embodiments, 5′-tiRNA is 5′-ti-Cys-GCA-10-1 (SEQ ID NO: 2).

In another aspect, the invention features a lipid nanoparticle including a 5′-tiRNA. In some embodiments, the 5′-tiRNA is between 30-37 nucleotides and includes nucleotides capable of forming a tRNA D-arm. In other embodiments, the 5′-tiRNA is modified (e.g., the 5′-tiRNA includes a non-natural or modified nucleoside or nucleotide). Exemplary modifications are chosen from 2′-O-methyl (2′-O-Me) modified nucleoside, a phosphorothioate (PS) bond between nucleosides; and a 2′-fluoro (2′-F) modified nucleoside. In some embodiments, the 5′-tiRNA has sequence identity to 5′-ti-Pro-CGG-1-1: GGCUCGUUGGUCUAGGGGUAUGAUUCUCGCUUCG (SEQ ID NO: 1). In embodiments, the 5′-tiRNA is 5′-ti-Pro-CGG-1 (SEQ ID NO: 1). In other embodiments, the 5′-tiRNA has sequence identity to 5′-ti-Cys-GCA-10-1: GGGGGUAUAGCUCAGGGGUAGAGCAUUUGACUG (SEQ ID NO: 2). In still other embodiments, 5′-tiRNA is 5′-ti-Cys-GCA-10-1 (SEQ ID NO: 2). In other embodiments, the lipid nanoparticle includes two or more 5′-tiRNAs. In embodiments, the lipid nanoparticles include two 5′-tiRNAs, wherein the first 5′-tiRNA has sequence identity to 5′-ti-Pro-CGG-1-1: GGCUCGUUGGUCUAGGGGUAUGAUUCUCGCUUCG (SEQ ID NO: 1) and the second 5′-tiRNA has sequence identity to 5′-ti-Cys-GCA-10-1: GGGGGUAUAGCUCAGGGGUAGAGCAUUUGACUG (SEQ ID NO: 2). In some embodiments, the two 5′-tiRNAs are 5′-ti-Pro-CGG-1-1: (SEQ ID NO: 1) and 5′-ti-Cys-GCA-10-1 (SEQ ID NO: 2).

In yet other aspect, the invention features an engineered cell including any of the aforementioned 5′-tiRNAs. In some embodiments, the cell includes two or more 5′-tiRNAs. Exemplary cells include an induced pluripotent stem cell (iPSC)-derived hematopoietic stem and progenitor cells (HSPC), a HSPC (e.g., from a donor), a myeloid progenitor cell, a lymphoid progenitor cell, or a granulocyte-macrophage progenitor (GMP). In some embodiments, the cell is autologous. In other embodiments, cell is banked.

In still another aspect, the invention features a treatment method including the step of: transfecting a cell, in a subject, with any of the aforementioned 5′-tiRNAs or contacting a cell, in a subject, with any of the aforementioned lipid nanoparticles under conditions effective to treat the subject.

In still another aspect, the invention features a treatment method including the step of: transplanting any one of aforementioned cells into a subject under conditions effective to treat a subject. In embodiments, the method treats a disease or disorder (e.g., a microbial infection, a fungal infection, a viral infection, a bacterial infection and the like). In embodiments, the disease or disorder is sepsis. In other embodiments, the treatment increases the number of neutrophils, granulocytes or macrophages in the subject. In still other embodiments, the treatment increases myeloid cell production in vivo. In other embodiments, the treatment is post-surgically administered. In other embodiments, treatment is administered to treat a trauma. In other embodiments, the treatment increases reconstitution of recovery after a stem cell transplant, after radiation therapy, or after a chemical injury to bone marrow. In other embodiments, the transplant is autologous or is allogenic.

In another aspect, the invention features composition including any one of the aforementioned 5′-tiRNAs. Such are typically formulated in a liposome, an exosome, or a lipid nanoparticle. In other embodiments, the composition includes any of the aforementioned engineered cells. In other embodiments, the composition is a pharmaceutical composition.

In another aspect, the invention features a method of increasing myeloid cell production in a subject, the method including: administering to the subject a therapeutically effective amount of any of the aforementioned compositions.

In another aspect, the invention features a method for modulating the differentiation of a stem-progenitor cell (SPC), including transfecting a stem-progenitor cell with one or more of the aforementioned 5′-tiRNAs. In embodiments, the stem-progenitor cells are induced pluripotent stem cells (iPSC). In embodiments, the stem-progenitor are hematopoietic stem-progenitor cells (HSPC). In embodiments, the stem-progenitor cells are granulocyte-macrophage progenitor cells (GMP). In embodiments, the stem-progenitor cells are isolated from a subject. In embodiments, the stem-progenitor cells are peripheral blood stem-progenitor cells. In embodiments, the 5′-tiRNA is formulated in an exosome, a liposome, or a lipid nanoparticle.

In another aspect, the invention features a method of delivering a 5′-tiRNA to an induced pluripotent stem cell (iPSC) or an iPSC population, the method including: a.) transfecting the iPSC or the iPSC population with any of the aforementioned 5′-tiRNAs in vitro; and b.) optionally, culturing the iPSC or the iPSC population in vitro; thereby delivering the 5′-tiRNA to the iPSC or the iPSC population. In embodiments, the method further includes culturing the transfected iPSC or the iPSC population. In embodiments, the iPSC or the iPSC population is autologous. In embodiments, the iPSC or the iPSC population is banked.

In another aspect, the invention features a method of delivering a 5′-tiRNA to a hematopoietic stem and/or progenitor cell (HSPC) or an HSPC population, the method including: a.) transfecting the HSPC or the HSPC population with any one of the aforementioned 5′-tiRNA in vitro; and b.) optionally, culturing the HSPC or the HSPC population in vitro; thereby delivering the 5′-tiRNA to the HSPC or the HSPC population.

In other aspects, the invention features an iPSC or iPSC population, an HSPC or HSPC population, an iPSC-derived HPSC, a GMP, a lymphoid progenitor cell, or a myeloid progenitor cell, each transfected with any one of the 5′-tiRNAs described herein.

In still another aspect, the invention features a method of treating a disease or disorder in a subject, the method including administering a therapeutically effective amount of a 5′-tiRNA to treat the disease or disorder in the subject.

In embodiments, the disease or disorder is an infection (e.g., a fungal (Candida) or bacterial infection).

In embodiments, the infection is a deep tissue infection.

In other embodiments, the disease or disorder is sepsis.

In embodiments, the 5′-tiRNA increases the number of neutrophils, granulocytes or macrophages in the subject to treat the disease or disorder.

In embodiments, the 5′-tiRNA increases myeloid cell production in the subject to treat the disease or disorder.

In embodiments, the 5′-tiRNA is post-surgically administered to treat the disease or disorder.

In embodiments, the 5′-tiRNA is administered to treat a trauma.

In embodiments, the 5′-tiRNA increases reconstitution or recovery after a stem cell transplant (e.g., autologous or allogeneic), after radiation therapy, or after a chemical injury to bone marrow.

In embodiments, the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

In embodiments, the 5′-tiRNA is intravenously administered.

In embodiments, the 5′-tiRNA is formulated in a liposome, an exosome, or a lipid nanoparticle.

In embodiments, the liposome, exosome, or lipid nanoparticle is intravenously administered.

In embodiments, the 5′-tiRNA is present in a cell which is administered to treat a disease or disorder in the subject.

In embodiments, the cell is an induced pluripotent stem cells (iPSC)-derived hematopoietic stem and progenitor cells (HSPC), a HSPC, a myeloid progenitor cell, a lymphoid progenitor cell, or a granulocyte-macrophage progenitor (GMP).

In another aspect, the invention features a method of delivering a 5′-tiRNA to a hematopoietic stem and/or progenitor cell (HSPC), the method including: a.) transfecting the HSPC with a 5′-tiRNA in vitro; and b.) optionally, culturing the HSPC in vitro; thereby delivering the 5′-tiRNA to the HSPC.

In embodiments, the HSPC is an iPSC-derived HSPC, an HSPC from a subject, a myeloid progenitor cell, a lymphoid progenitor cell, or a GMP.

In embodiments, the HSPC is a human cell or sample.

In embodiments, the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

In another aspect, the invention features an HSPC transfected with a 5′-tiRNA.

In embodiments, the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

In embodiments, the HSPC is autologous with respect to a patient to be administered the cell.

In embodiments, the HSPC is allogenic with respect to a patient to be administered the cell.

In another aspect, the invention features an HSPC produced according to the aforementioned methods.

In embodiments, the HSPC is an iPSC-derived HSPC, an HSPC from a subject, a myeloid progenitor cell, a lymphoid progenitor cell, or a GMP.

Other features and advantages of the invention will be apparent from the following Detailed Description and the Claims.

### BRIEF DESCRIPTION OF THE DRAWINGS

The present disclosure contains at least one drawing executed in color. Copies of this patent or patent application publication with color drawing(s) will be provided by the Office upon request and payment of the necessary fee.

For a more complete understanding of the principles disclosed herein, and the advantages thereof, reference is now made to the following descriptions taken in conjunction with the accompanying drawings.

FIG. 1A shows a schematic illustrating the investigation of EV-mediated transfer of stromal-derived sncRNAs using lethally irradiated reporter mice that express GFP in specific mesenchymal subsets at different stages of differentiation and transplanted with congenic CD45.1 bone marrow cells.

FIG. 1B shows the frequency of GFP+ cells in donor CD45.1+ BM (parent gate). Data represent independent biological replicates. Data is presented as mean±SD. *p<0.05, **p<0.01, and ****p<0.0001.

FIG. 1C shows imaging flow cytometry (IFC) on sorted LKS^{GFP+/-} cells from Ocn-GFP^{Topaz} animals. Scale bar represents 3 μm.

FIG. 1D shows confocal imaging on IFC-sorted LKS^{GFP+/-} cells from Ocn-GFP^{Topaz} animals. Scale bar represents 3 μm.

FIG. 1E shows the differential transfer of PKH-26-labeled EVs from MSCs or osteoblasts to co-cultured GMPs as shown by flow cytometry. Gates are on live cells. Data is presented as mean±SD. *p<0.05, **p<0.01, and ****p<0.0001.

FIG. 1F show the number of methyl cellulose colonies in osteoblasts compared to LKS^{GFP+}; n=2.

FIG. 1G shows transmission electron microscopy of BM-derived EVs. Scale bars represent 100 nm.

FIG. 1H shows an immunogold staining using 15-nm gold beads (TSG-101) and 10-nm gold beads (GFP). Scale bars represent 100 nm.

FIG. 1I shows a nanoparticle tracking analysis (NTA) illustrating the size distribution of EVs isolated from the mouse BM. The mean and mode are calculated based on 5 measurements.

FIG. 1J shows a western blot analysis for TSG101 and GFP on EVs and cellular lysates.

FIG. 1K shows a GFP-targeted qPCR on RNA extracted from RNase-A-treated EVs. Data represent three technical replicates.

FIG. 2A shows the frequency of GFP+ cells (of parent gate) within BM HSPCs. Data represent independent biological replicates. Data is presented as mean±SD. **p<0.01, ***p<0.001, and ****p<0.0001.

FIG. 2B shows an imaging flow cytometry (IFC) of sorted GMP^{GFP+} and GMP^{GFP-}. Scale bar represents 3 μm.

FIG. 2C shows confocal imaging of IFC-sorted GMP^{GFP+} and GMP^{GFP-}. Scale bar represents 3 μm.

FIG. 2D shows a morphological assessment of GMP^{GFP+} by flow cytometry. Scale bar represents 10 μm.

FIG. 2E shows a morphological assessment of GMP^{GFP+} by bright-field microscopy of Wright Giemsa staining. Scale bar represents 10 μm.

FIG. 2F shows the number of hematopoietic colonies in methyl cellulose comparing GMP^{GFP+} to GMP^{GFP-}. Statistical significance is calculated using paired Student's t test; *p<0.05. Data represent one out of three independent experiments.

FIG. 2G shows confocal imaging of GMP (labeled with CFP) cells with PKH-26-labeled vesicles (yellow vesicles+white arrows). Top image: XYZ view of GMP (CFP) cell with PKH-26-labeled vesicles (yellow vesicles+white arrows). Scale bar represents 5 μm. Bottom three images: maximum projection by confocal imaging of live osteoblast (GFP) and GMP (CFP) co-culture demonstrating the transfer of PKH-26-labeled vesicles (yellow+white arrows) from osteoblasts to GMPs as indicated by the white arrows is shown. Scale bar represents 10 μm.

FIG. 2H shows the frequency of live progenitors labeled with PKH-26 vesicles from co-cultured osteoblasts. Data is presented as mean±SD. **p<0.01, ***p<0.001, and ****p<0.0001.

FIG. 2I shows a fluorescence-activated cell sorting (FACS) analysis of BMof Ocn-GFP mice injected with pHrodo; percentages are of parent gate granulocytic (Ly6G+) and monocytic (Ly6G−Ly6C+) cells gated on non-erythroid (CD71−Ter119−) BM (J) GMP^{GFP+} and GMP^{GFP-}.

FIG. 2J shows a fluorescence-activated cell sorting (FACS) analysis of BMof Ocn-GFP mice injected with pHrodo; percentages are of parent gate GMP^{GFP+} and GMP^{GFP-}.

FIG. 2K shows the fold change in GFP+ cells post-irradiation, 5FU, and systemic C. albicans infection. Fold change is calculated from the mean of GFP+ cells frequency of two independent experiments as shown in FIGS. 8I-N.

FIG. 3A shows an overview of RNA sequencing experiment using Ocn-GFP animals.

FIG. 3B shows fractions of small RNA sequencing reads mapped to genomic elements in BM EVs.

FIG. 3C shows the top ten tRNAs ranked by their abundance in BM EVs. Data represent three biological replicates.

FIG. 3D shows fractions of small RNA sequencing reads mapped to genomic elements in GMP^{GFP+} and GMP^{GFP-}. Data represent 7 biological replicates.

FIG. 3E shows the percent of reads mapping to tRNAs in GMP^{GFP+} and GMP^{GFP-}. Data are presented as mean±SD. **p<0.01.

FIG. 3F shows a principal-component analysis (PCA) based on tRNAs expression in GMP^{GFP+} and GMP^{GFP-}.

FIG. 3G shows a heatmap of tRNAs that are more abundant in GMP^{GFP+}; >1.5-fold change. The levels are shown as relative to the average abundance of a given tRNA across all samples. Given extremely high sequence similarity between tRNA species sharing the same anticodon (FIG. 9), one individual tRNA representative per group is used. Data represent one of two independent experiments.

FIG. 3H shows a sybr gold-stained RNA gel with 750 ng total RNA per sample (left image) and a northern blot analysis of small RNAs collected from total GMPs (labeled as G) and BM EVs (labeled as E) (right image).

FIG. 3I shows the transfer of synthetic Cy3-labeled 5′-ti-Pro-CGG-1 from transfected primary osteoblasts to co-cultured GMPs.

FIG. 3J shows a PCA of transcriptome-wide gene expression levels in GMP^{GFP+} and GMP^{GFP-}, based on mRNA sequencing.

FIG. 3K show GSEA enrichment plots for ribosomal and translation-related genes.

FIG. 3L shows the top gene sets enriched in GFP+ cells according to GSEA.

FIG. 3M shows a PCA based on tRNA expression in control and irradiated BM EVs.

FIG. 3N shows a heatmap of tRNAs with >1.5-fold change comparing GMP^{GFP+} to GMP^{GFP-}, in 2 Gy irradiated Ocn-GFP mice. The levels are shown as relative to the average abundance of a given tRNA across all samples. Given extremely high sequence similarity between tRNA species sharing the same anticodon (FIG. 9), one individual tRNA representative per group is used.

FIG. 4A shows an analysis of EV-labeled GMPs (GMP^{GFP+}) for the incorporation of OPP. Data represent two independent experiments,

FIG. 4B shows an analysis of EV-labeled GMPs (GMP^{GFP+}) for the incorporation of OPP. Data represent two independent experiments. Data is presented as mean±SD. *p<0.05, **p<0.01, ***p<0.001, and ****p<0.0001.

FIG. 4C shows a gene set enrichment analysis (GSEA) of EV-labeled GMPs (GMP^{GFP+}), n=3. Data represent one of two independent experiments.

FIG. 40 shows a cell cycle analysis of EV-labeled GMPs (GMP^{GFP+}), n=3. Data represent one of two independent experiments. Data is presented as mean±SD. *p<0.05, **p<0.01, ***p<0.001, and ****p<0.0001.

FIG. 4E shows an analysis of a clonally derived myeloid cell line's (HoxA9) ability to incorporate OPP; analysis was performed using a paired Student's t test, n=4. Data represent one of 2 independent experiments.

FIG. 4F shows a cell cycle analysis of a clonally derived myeloid cell line (HoxA9); analysis was performed using a paired Student's t test, n=4. Data represent one of 2 independent experiments. Data is presented as mean±SD. *p<0.05, **p<0.01, ***p<0.001, and ****p<0.0001.

FIG. 4G shows a schematic illustrating an experimental outline for assaying the uptake of PKH-26-labeled BM EVs by live GMPs in culture.

FIG. 4H shows an imaging flow cytometry analysis illustrating the uptake of PKH-26-labeled BM EVs by live GMPs in culture.

FIG. 4I shows enhanced OPP incorporation in GMPs that take up PKH-26-labeled EVs. Data is presented as mean±SD. *p<0.05, **p<0.01, ***p<0.001, and ****p<0.0001.

FIG. 4J shows enhanced cellular proliferation in GMPs that take up PKH-26-labeled EVs.

FIG. 5A shows OPP incorporation of synthetic Cy3-labeled tiRNA (Pro-CGG-1) or control piRNA-transfected GMPs. Analysis is performed on live, Cy3+ cells, n=6. Data represent two independent experiments. Data is presented as mean±SD. *p<0.05, **p<0.01, ***p<0.001, and ****p<0.0001.

FIG. 5B shows a cell cycle analysis of synthetic Cy3-labeled tiRNA (Pro-CGG-1) or control piRNA-transfected GMPs. Analysis is performed on live, Cy3+ cells, n=6. Data represent two independent experiments. Data is presented as mean±SD. *p<0.05, **p<0.01, ***p<0.001, and ****p<0.0001.

FIG. 5C shows OPP incorporation of synthetic Cy3-labeled tiRNA (Cys-GCA-27) or control piRNA-transfected GMPs. Analysis is performed on live, Cy3+ cells, n=6. Data represent two independent experiments. Data is presented as mean±SD. *p<0.05, **p<0.01, ***p<0.001, and ****p<0.0001.

FIG. 5D shows a cell cycle analysis of synthetic Cy3-labeled tiRNA (Cys-GCA-27) or control piRNA-transfected GMPs. Analysis is performed on live, Cy3+ cells, n=6. Data represent two independent experiments. Data is presented as mean±SD. *p<0.05, **p<0.01, ***p<0.001, and ****p<0.0001.

FIG. 5E shows the YFP intensity in TOP-H2B-YFP-DD reporter transduced and tiRNA transfected GMPs (labeled GMP-TOP). Cells were treated with 10 μM TMP 12 hrs before analysis. Data is presented as mean±SD. *p<0.05, **p<0.01, ***p<0.001, and ****p<0.0001. Statistical analysis is calculated using one-way ANOVA.

FIG. 5F shows the YFP intensity in IRES-H2B-YFP-DD reporter transduced and tiRNA transfected GMPs (labeled GMP-IRES). Cells were treated with 10 μM TMP 12 hrs before analysis. Data is presented as mean±SD. *p<0.05, **p<0.01, ***p<0.001, and ****p<0.0001. Statistical analysis is calculated using one-way ANOVA.

FIG. 5G shows the YFP intensity in TOP-H2B-YFP-DD reporter transduced and tiRNA transfected LKS cells (labeled LKS-TOP). Cells were treated with 10 μM TMP 12 hrs before analysis. Data is presented as mean±SD. *p<0.05, **p<0.01, ***p<0.001, and ****p<0.0001. Statistical analysis is calculated using one-way ANOVA.

FIG. 5H shows the YFP intensity in IRES-H2B-YFP-DD reporter transduced and tiRNA transfected LKS cells (labeled LKS-IRES). Cells were treated with 10 μM TMP 12 hrs before analysis. Data is presented as mean±SD. *p<0.05, **p<0.01, ***p<0.001, and ****p<0.0001. Statistical analysis is calculated using one-way ANOVA.

FIG. 6A shows a representative image of a phenotypic analysis by flow cytometry of 5′-ti-Pro-CGG-1 or piRNA control transfected GMPs; gates are on Cy3+, CD11b+, CX3CR1+ cells.

FIG. 6B shows a representative image of a phenotypic analysis by flow cytometry of 5′-ti-Pro-CGG-1 or piRNA control transfected GMPs; gates are on Ly6g+ and CXCR2+ cells.

FIG. 6C shows a quantification of the phenotypic analysis of FIG. 6A. Data represent two independent experiments. Data is presented as mean±SD. *p<0.05, **p<0.01 ***p<0.001, and ****p<0.0001.

FIG. 6D shows a quantification of the phenotypic analysis of FIG. 6B. Data represent two independent experiments. Data is presented as mean±SD. *p<0.05, **p<0.01 ***p<0.001, and ****p<0.0001.

FIG. 6E shows a representative image of a phagocytosis assay analysis by flow cytometry. Gates are on Cy3+ cells. Data represent two independent experiments.

FIG. 6F shows a quantification of the phagocytosis assay analysis of FIG. 6E. Data represent two independent experiments. Data is presented as mean±SD. *p<0.05, **p<0.01 ***p<0.001, and ****p<0.0001.

FIG. 6G shows the fluorescence signal from metabolically active C. albicans co-cultured with Cy3+ GMPs for 2 h. Data represent one of two independent experiments. Analysis was performed using one-way ANOVA with no correction for multiple comparisons. Data is presented as mean±SD. *p<0.05, **p<0.01 ***p<0.001, and ****p<0.0001.

FIG. 6H shows the frequency of GMP^{GFP+} (parent gate) after 14 days of iPTH injections. Data represent independent biological replicates of two independent experiments. Data is presented as mean±SD. *p<0.05, **p<0.01 ***p<0.001, and ****p<0.0001.

FIG. 6I shows a quantification of peripheral blood neutrophils (Ly6g+) in iPTH-treated mice 14 days post-irradiation. Data represent two independent experiments. Data is presented as mean±SD. *p<0.05, **p<0.01 ***p<0.001, and ****p<0.0001.

FIG. 6J shows a quantification of peripheral blood monocytes (Ly6c+) in iPTH-treated mice 14 days post-irradiation. Data represent two independent experiments. Data is presented as mean±SD. *p<0.05, **p<0.01 ***p<0.001, and ****p<0.0001.

FIG. 6K shows peripheral blood white blood cell (WBC) counts in caPPR mice infected with C. albicans.

FIG. 6L shows peripheral blood neutrophil counts (Ly6G+) in caPPR mice infected with C. albicans.

FIG. 6M shows a survival analysis in caPPR mice post C. albicans infection. Data represent one of two independent experiments.

FIG. 7A shows the gating strategy for the detection of CD45.1+GFP+ BM cells.

FIG. 7B shows the frequency of GFP+ mesenchymal cells in non-hematopoietic, non-endothelial bone cells. Data is presented as mean±s.d.

FIG. 7C shows the frequency of CD45+GFP+ BM cells in transplanted and non-transplanted Ocn-GFP animals. Data is presented as mean±s.d.

FIG. 7D shows the gating strategy for LKS^{GFP+} sorted for imaging flow cytometry and confocal microscopy.

FIG. 7E shows the gating strategy for CD45− GFP+ osteoblasts and CD45+ GFP+ LKS sorted for colony forming assay.

FIG. 7F shows an image of hematopoietic colonies in methyl cellulose; images are acquired using 4× objective.

FIG. 7G shows an imaging flow cytometry that reveals that LKS^{GFP+} methyl cellulose colonies are GFP− as compared to GFP+ osteoblasts.

FIG. 7H shows a qPCR quantification that reveals that LKS^{GFP+} methyl cellulose colonies are GFP− as compared to GFP+ osteoblasts.

FIG. 7I shows a schematic representation of the flowcytometry assay (upper panel). Briefly, streptavidin beads are coated with EVs bound to biotinylated anti-CD81 and then labeled using anti CD9-AF647. The lower panel illustrates a representative image of the flow cytometry analysis of bead-captured EVs.

FIG. 7J shows a quantification of the relative expression of GFP by qPCR in RNA extracted from GMPs cultured with or without Ocn-GFP BM EVs. Data represent three technical replicates. Data is presented as mean±s.d.

FIG. 8A shows the gating strategy of GFP labeled BM HSPC populations. Parent gates are indicated above the plots (upper) and to the left of the plots (lower).

FIG. 8B shows negligible labeling of SLAM HSCs by Ocn-GFP^{Topaz} BM derived EVs.

FIG. 8C shows a quantification of the percentage of GFP+ labeling of HSPCs by osteoblast-derived EVs in the Col1-GFP reporter model. Percentages are of parent gate. Data represent independent biological replicates. Data is presented as mean±s.d. *p<0.05, **p<0.01, ***p<0.001.

FIG. 8D shows a representative flow cytometry image of labeled HSPCs by osteoblast-derived EVs in the Col1-GFP reporter model. Percentages are of parent gate.

FIG. 8E shows a maximum projection by confocal imaging of live GMPs demonstrating lack of PKH-26 labeling in the absence of PKH labeled osteoblasts. Scale bar=15 μm.

FIG. 8F shows a quantification of the area of GMP^{GFP-} and GMP^{GFP+} colonies grown on methyl cellulose, as measured by ImageJ. Data represent 6 independent biological replicates with at least 10 colonies representing each replicate. Data is presented as mean±s.d. *p<0.05, **p<0.01, ***p<0.001.

FIG. 8G shows a quantification of GFP+ cells illustrating that osteoblast derived EVs label mature cells in the BM. Percentages are of parent gate. Data represent independent biological replicates. Data is presented as mean±s.d. *p<0.05, **p<0.01, ***p<0.001.

FIG. 8H shows representative flow cytometry images of osteoblast derived EVs labeling mature cells in the BM. Percentages are of parent gate.

FIG. 8I shows the frequency of GMP^{GFP+} in total BM mononuclear cells following lose-dose radiation (2Gy). Data is presented as mean±s.d. *p<0.05, **p<0.01, ***p<0.001.

FIG. 8J shows the frequency of GMP^{GFP+} in total BM mononuclear cells following 5-fluorouracil (5FU) administration. Data is presented as mean±s.d. *p<0.05, **p<0.01, ***p<0.001.

FIG. 8K shows the frequency of GMP^{GFP+} in total BM mononuclear cells following C. albicans infection. Data is presented as mean±s.d. *p<0.05, **p<0.01, ***p<0.001.

FIG. 8L shows the frequency of CMP^{GFP+} and LKS^{GFP+} in total BM live mononuclear cells post-stress with low-dose radiation (2Gy). Data is presented as mean±s.d. *p<0.05, **p<0.01, ***p<0.001.

FIG. 8M shows the frequency of CMP^{GFP+} and LKS^{GFP+} in total BM live mononuclear cells post-stress with 5-fluorouracil (5FU) administration. Data is presented as mean±s.d. *p<0.05, **p<0.01, ***p<0.001.

FIG. 8N shows the frequency of CMP^{GFP+} and LKS^{GFP+} in total BM live mononuclear cells post-stress with C. albicans systemic infection. Data is presented as mean±s.d. *p<0.05, **p<0.01, ***p<0.001.

FIG. 8O shows the absolute number of GMPs in live mononuclear cells 12 hrs post stress with low-dose radiation (2Gy). Data is presented as mean±s.d. *p<0.05, **p<0.01, ***p<0.001.

FIG. 8P shows the absolute number of GMPs in live mononuclear cells 12 hrs post stress with 5-fluorouracil (5FU) administration. Data is presented as mean±s.d. *p<0.05, **p<0.01, ***p<0.001.

FIG. 8Q shows the absolute number of GMPs in live mononuclear cells 12 hrs post stress with C. albicans systemic infection. Data is presented as mean±s.d. *p<0.05, **p<0.01, ***p<0.001.

FIG. 8R shows the absolute number of GMPs in live mononuclear cells 12 and 24 hrs post C. albicans infection. Data represent two independent experiments. Data is presented as mean±s.d. *p<0.05, **p<0.01, ***p<0.001.

FIG. 8S shows the absolute number of CMPs in live mononuclear cells 12 and 24 hrs post C. albicans infection. Data represent two independent experiments. Data is presented as mean±s.d. *p<0.05, **p<0.01, ***p<0.001.

FIG. 8T shows the absolute number of LKS in live mononuclear cells 12 and 24 hrs post C. albicans infection. Data represent two independent experiments. Data is presented as mean±s.d. *p<0.05, **p<0.01, ***p<0.001.

FIG. 9 shows the distributions of density of mapped sequencing reads across the length of tRNA sequences with differential abundance between GMP^{GFP+} vs GMP^{GFP-}, shown for BM-EVs, GMP^{GFP+}, GMP^{GFP-}, osteoblast EVs, and osteoblasts. The sequence of a single tRNA representative is shown for each group of highly similar tRNA species (data not shown). The density of reads (CPM) at each tRNA position is shown by shading.

FIG. 10A shows the levels of the ten most abundant miRNAs detected in BM EVs, represented as read counts per million (CPM). Data represents three independent biological replicates.

FIG. 10B shows the fractions of small RNA sequencing reads mapped to genomic elements in osteoblast EVs (upper) and osteoblasts (Lower). Data represents three biological replicates.

FIG. 10C shows the percentage of total reads for the indicated small RNA fractions in GMP^{GFP+} and GMP^{GFP-}.

FIG. 10D shows a heatmap of tRNAs that are more abundant in GMP^{GFP+} cells >1.5 fold difference compared to GMP^{GFP-} cells. The levels are shown as relative to the average abundance of a given tRNA across all samples. Data represents one of two independent experiments.

FIG. 10E shows a heatmap of the tRNA set shown in FIG. 10D, comparing the levels of these tRNAs in osteoblasts versus osteoblast EVs and BM EVs. The levels are shown as relative to the average abundance of a given tRNA across all samples.

FIG. 10F shows a northern blot analysis of small RNAs collected from BM CD45+/− cells and BM EVs (left image); and a SYBR gold stained RNA gel (right image). 500 ng Total RNA was loaded.

FIG. 10G shows a heatmap of expression levels of the genes differentially expressed between GMP^{GFP+} and GMP^{GFP-} cells (>2-fold change, FDR <0.001). Expression levels are shown as relative to the average for a given gene across all samples.

FIG. 11A shows the gating strategy for cell cycle analysis of GMP^{GFP+} and GMP^{GFP-}.

FIG. 11B shows a cell cycle analysis of clonally derived myeloid cell line labeled with EVs.

FIG. 11C shows OPP uptake in Cy3 labeled transfected tiRNA in primary GMPs.

FIG. 11D shows the gating strategy for cell cycle analysis in Cy3 labeled transfected tiRNA in primary GMPs.

FIG. 11E shows the OPP uptake of tiRNA transfected GMPs, n=6. Data represent two independent experiments.

FIG. 11F shows a cell cycle analysis of tiRNA transfected GMPs, n=6. Data represent two independent experiments. Data is presented as mean±s.d. **p<0.01, ****p<0.0001.

FIG. 11G shows a Sybr gold-stained RNA gel loaded with 75 ng total RNA for EVs (E) and media (M) samples and 2 μg for the Osteoblast (O) sample (left image). Also shown is a northern blot analysis of small RNAs collected from osteoblasts and their EVs released in the culture media (right image). Data is presented as mean±s.d. **p<0.01, ****p<0.0001.

FIG. 12A shows a one-way ANOVA analysis results of FIG. 6G.

FIG. 12B shows the frequency of GFP+ osteoblasts (parent gate) within Ter119 CD45− CD31− bone cells 14 days post iPTH treatment. Data represent independent biological replicates from two independent experiments.

FIG. 12C shows a flow plot demonstrating an increased in GMP^{GFP+} 14 days post iPTH treatment. Percentages are of parent gate.

FIG. 12D shows the frequency of GFP+ osteoblasts (parent gate) within Ter119 CD45− CD31− bone cells 14 days post iPTH treatment. Data represents one experiment and is presented as mean±s.d. *p<0.05, **p<0.01, ****p<0-0001.

FIG. 12E shows an increase of GMP^{GFP+} in caPPR mice. Percentages are of parent gate. Data represents one experiment and is presented as mean±s.d. *p<0.05, **p<0.01, ****p<0-0001.

FIG. 12F shows peripheral blood WBC counts in C. albicans infected caPPR mice. Data represents one experiment and is presented as mean±s.d. *p<0.05, **p<0.01, ****p<0-0001.

FIG. 12G shows peripheral blood neutrophil counts in C. albicans infected caPPR mice. Data represents one experiment and is presented as mean±s.d. *p<0.05, **p<0.01, ****p<0-0001.

FIG. 12H shows a survival analysis in caPPR mice post C. albicans infection. Data represent one of two independent experiments.

FIG. 13A shows a representative image of a phenotypic analysis by flow cytometry of 5′-ti-Cys-GCA-27 or piRNA control transfected GMPs; gates are on Cy3+, CD11b+, CXCR2+ cells.

FIG. 13B shows a representative image of a phenotypic analysis by flow cytometry of 5′-ti-Cys-GCA-27 or piRNA control transfected GMPs; gates are on CD11b+ and CX3CR1+ cells.

FIG. 13C shows a quantification of the phenotypic analysis of FIG. 13A. Data represent two independent experiments. Data is presented as mean±SD. *p<0.05, ****p<0.0001.

FIG. 13D shows a quantification of the phenotypic analysis of FIG. 13B. Data represent two independent experiments. Data is presented as mean±SD. *p<0.05, ****p<0.0001.

### DETAILED DESCRIPTION

Unless defined otherwise, all technical and scientific terms used have the same meaning as commonly understood by one of ordinary skill in the art to which this invention belongs. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA or as RNA, as specified; disclosure of one necessarily defines the other, as is known to one of ordinary skill in the art.

The term “comprise” is intended to mean “include”. Where a term is provided in the singular, it also contemplates aspects of the invention described by the plural of that term. The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The following disclosure, as is discussed below, provides, inter alia, 5′-tiRNA molecules and various modifications thereof, as well as cells and compositions including such molecules. This disclosure also provides various methods of making and using these molecules, cells and compositions. Methods of administering and treating subjects (e.g., humans) with 5′-tiRNAs, such as methods of transfecting and transplanting the 5′-tiRNAs also described herein.

5′-tiRNAs

In one aspect, the disclosure relates to 5′-tiRNAs, which are truncated forms of tRNAs. Such 5′-tiRNAs are processed from their cognate tRNAs and typically are about 30-35 nucleotides in length and may be naturally- or non-naturally occurring. In embodiments, the 5′-tiRNAs useful in the compositions and methods described herein are synthetic, being produced according to standard methods known in the art such as those described herein. In embodiments, the 5′-tiRNA molecule is between 23-37 nucleotides in length. The 5′-tiRNA molecule may further include modifications as described herein, The 5′-tiRNA may further include a nucleotide sequence corresponding to the D-arm, or a portion thereof, of the tRNA molecule (e.g., a human tRNA). In various other embodiments, the 5′-tiRNA does not include an anticodon or alternatively includes a partial anticodon.

The following table provides human 5′-tiRNAs useful in producing the various compositions, cells, and methods described herein.

TABLE 1

| | | SEQ |
|---|---|---|
| Name | tiRNA sequence | ID |
| 5′-ti-Pro- | GGCUCGUUGGUCUAGGG | 1 |
| CGG-1-1 | GUAUGAUUCUCGCUUAG | |
| 5′-ti-Cys- | GGGGGUAUAGCUCAGGG | 2 |
| GCA-10-1 | GUAGAGCAUUUGACUG | |
| 5′-ti-Cys- | GGGCGUAUAGCUCAGGG | 3 |
| GCA-22-1 | GUAGAGCAUUUGACUG | |
| 5′-ti-Cys- | GGGGAUAUAGCUCAGGG | 4 |
| GCA-17-1 | GUAGAGCAUUUGACUG | |
| 5′-ti-Cys- | GGGGGUAUAGCUCAGGG | 5 |
| GCA-3-1 | GUAGAGCACUUGACUG | |
| 5′-ti-Cys- | GGGGGUAUAGCUCAGUG | 6 |
| GCA-4-1 | GUAGAGCAUUUGACUG | |
| 5′-ti-Cys- | GGGGGUAUAGCUUAGGG | 7 |
| GCA-12-1 | GUAGAGCAUUUGACUG | |
| 5′-ti-Cys- | GGGGGUAUAGUUCAGGG | 8 |
| GCA-18-1 | GUAGAGCAUUUGACUG | |
| 5′-ti-Cys- | GGGGGUAUAGCUCAGGU | 9 |
| GCA-7-1 | GGUAGAGCAUUUGACUG | |
| 5′-ti-Cys- | GGGGGUAUAGCUCAGUG | 10 |
| GCA-5-1 | GGUAGAGCAUUUGACUG | |
| 5′-ti-Cys- | GGGGGCAUAGCUCAGUG | 11 |
| GCA-1-1 | GUAGAGCAUUUGACUG | |
| 5′-ti-Cys- | GGGGGUAUAGCUCACAG | 12 |
| GCA-23-1 | GUAGAGCAUUUGACUG | |
| 5′-ti-Cys- | GGGGGUAUAGCUUAGCG | 13 |
| GCA-11-1 | GUAGAGCAUUUGACUG | |
| 5′-ti-Cys- | GGGGGUGUAGCUCAGUG | 14 |
| GCA-6-1 | GUAGAGCAUUUGACUG | |
| 5′-ti-Cys- | GGGGGUAUAUCUCAGGG | 15 |
| GCA-25-1 | GGCAGAGCAUUUGACUG | |

Exemplary 5′-tiRNA molecules include a 5′-ti-Pro-CGG-1-1 (SEQ ID NO: 1) and a 5′-ti-Cys-GCA-10-1 (SEQ ID NO: 2), or other 5′-tiRNA molecules having sequence identity to these molecules.

By way of example, the 5′-ti-Pro-CGG-1-1 is typically 30-37 nucleotides in length (e.g., 30, 31, 32, 33, 34, 35, 36, or 37 nucleotides) and typically includes the nucleotides that create the D-arm of the corresponding tRNA. In other embodiments, the 5′-ti-Pro-CGG-1-1 may be shorter by way of a truncation on the 5′ and/or 3′ end. By way of example, SEQ ID NO: 1 may be truncated on the 5′ and/or 3′ end such that the 5′-tiRNA is less than 30 nucleotides in length (e.g., 29, 28, 27, 26, 25, 24, 23, or fewer nucleotides in length). Examples of these truncations are depicted below. A dash (-) indicates the truncation.

5′-ti-Pro-CGG-1-1:
(SEQ ID NO: 1)
GGCUCGUUGGUCUAGGGGUAUGAUUCUCGCUUCG
5′-ti-Pro-CGG-1-1:
(SEQ ID NO: 16)
-GCUCGUUGGUCUAGGGGUAUGAUUCUCGCUUCG
5′-ti-Pro-CGG-1-1:
(SEQ ID NO: 17)
--CUCGUUGGUCUAGGGGUAUGAUUCUCGCUUCG
5′-ti-Pro-CGG-1-1:
(SEQ ID NO: 18)
---UUGGUCUAGGGGUAUGAUUCUCGCUUCG
5′-ti-Pro-CGG-1-1:
(SEQ ID NO: 19)
----CGUUGGUCUAGGGGUAUGAUUCUCGCUUCG
5′-ti-Pro-CGG-1-1:
(SEQ ID NO: 20)
-----GUUGGUCUAGGGGUAUGAUUCUCGCUUCG
5′-ti-Pro-CGG-1-1:
(SEQ ID NO: 21)
GGCUCGUUGGUCUAGGGGUAUGAUUCUCGCUUC-
5′-ti-Pro-CGG-1-1:
(SEQ ID NO: 22)
GGCUCGUUGGUCUAGGGGUAUGAUUCUCGCUU--
5′-ti-Pro-CGG-1-1:
(SEQ ID NO: 23)
GGCUCGUUGGUCUAGGGGUAUGAUUCUCGCU---
5′-ti-Pro-CGG-1-1:
(SEQ ID NO: 24)
GGCUCGUUGGUCUAGGGGUAUGAUUCUCGC----
5′-ti-Pro-CGG-1-1:
(SEQ ID NO: 25)
GGCUCGUUGGUCUAGGGGUAUGAUUCUCG-----
5′-ti-Pro-CGG-1-1:
(SEQ ID NO: 26)
-GCUCGUUGGUCUAGGGGUAUGAUUCUCGCUUC-
5′-ti-Pro-CGG-1-1:
(SEQ ID NO: 27)
--CUCGUUGGUCUAGGGGUAUGAUUCUCGCUU--
5′-ti-Pro-CGG-1-1:
(SEQ ID NO: 28)
---UCGUUGGUCUAGGGGUAUGAUUCUCGCU---
5′-ti-Pro-CGG-1-1:
(SEQ ID NO: 29)
----CGUUGGUCUAGGGGUAUGAUUCUCGC----
5′-ti-Pro-CGG-1-1:
(SEQ ID NO: 30)
-----GUUGGUCUAGGGGUAUGAUUCUCG-----

In embodiments, the 5′-ti-Pro-CGG-1-1 may be 35, 36, or 37 nucleotides in length, respectively:
5′-ti-Pro-CGG-1-1: GGCUCGUUGGUCUAGGGGUAUGAUUCUCGCUUCGG (SEQ ID NO: 31)
5′-ti-Pro-CGG-1-1: GGCUCGUUGGUCUAGGGGUAUGAUUCUCGCUUCGGG (SEQ ID NO: 32)
5′-ti-Pro-CGG-1-1: GGCUCGUUGGUCUAGGGGUAUGAUUCUCGCUUCGGGU (SEQ ID NO: 33)

In still other embodiments, the 5′-ti-Cys-GCA-10-1 is 30-37 nucleotides in length (e.g., 30, 31, 32, 33, 34, 35, 36, or 37 nucleotides) and typically includes the nucleotides that create the D-arm of the corresponding tRNA. In still other embodiments, the 5′-ti-Cys-GCA-10-1 may be shorter by way of a truncation on the 5′ and/or 3′ end. By way of example, SEQ ID NO: 2 may be truncated on the 5′ and/or 3′ end such that the 5′-tiRNA is less than 30 nucleotides in length (e.g., 29, 28, 27, 26, 25, 24, 23, or fewer nucleotides in length). Examples of these truncations are depicted below. A dash (-) indicates the truncation.

5′-ti-Cys-GCA-10-1:
(SEQ ID NO: 2)
GGGGGUAUAGCUCAGGGGUAGAGCAUUUGACUG
5′-ti-Cys-GCA-10-1:
(SEQ ID NO: 34)
-GGGGUAUAGCUCAGGGGUAGAGCAUUUGACUG
5′-ti-Cys-GCA-10-1:
(SEQ ID NO: 35)
--GGGUAUAGCUCAGGGGUAGAGCAUUUGACUG
5′-ti-Cys-GCA-10-1:
(SEQ ID NO: 36)
---GGUAUAGCUCAGGGGUAGAGCAUUUGACUG
5′-ti-Cys-GCA-10-1:
(SEQ ID NO: 37)
----GUAUAGCUCAGGGGUAGAGCAUUUGACUG
5′-ti-Cys-GCA-10-1:
(SEQ ID NO: 38)
-----UAUAGCUCAGGGGUAGAGCAUUUGACUG
5′-ti-Cys-GCA-10-1:
(SEQ ID NO: 39)
GGGGGUAUAGCUCAGGGGUAGAGCAUUUGACU-
5′-ti-Cys-GCA-10-1:
(SEQ ID NO: 40)
GGGGGUAUAGCUCAGGGGUAGAGCAUUUGAC--
5′-ti-Cys-GCA-10-1:
(SEQ ID NO: 41)
GGGGGUAUAGCUCAGGGGUAGAGCAUUUGA---
5′-ti-Cys-GCA-10-1:
(SEQ ID NO: 42)
GGGGGUAUAGCUCAGGGGUAGAGCAUUUG----
5′-ti-Cys-GCA-10-1:
(SEQ ID NO: 43)
GGGGGUAUAGCUCAGGGGUAGAGCAUUU-----
5′-ti-Cys-GCA-10-1:
(SEQ ID NO: 44)
-GGGGUAUAGCUCAGGGGUAGAGCAUUUGACU-
5′-ti-Cys-GCA-10-1:
(SEQ ID NO: 45)
--GGGUAUAGCUCAGGGGUAGAGCAUUUGAC--
5′-ti-Cys-GCA-10-1:
(SEQ ID NO: 46)
---GGUAUAGCUCAGGGGUAGAGCAUUUGA---
5′-ti-Cys-GCA-10-1:
(SEQ ID NO: 47)
----GUAUAGCUCAGGGGUAGAGCAUUUG----
5′-ti-Cys-GCA-10-1:
(SEQ ID NO: 48)
-----UAUAGCUCAGGGGUAGAGCAUUU-----

In embodiments, the 5′-ti-Cys-GCA-1-1 may be 35, 36, or 37 nucleotides in length, respectively:

5′-ti-Cys-GCA-10-1:
(SEQ ID NO: 49)
GGGGGUAUAGCUCAGGGGUAGAGCAUUUGACUGC
5′-ti-Cys-GCA-10-1:
(SEQ ID NO: 50)
GGGGGUAUAGCUCAGGGGUAGAGCAUUUGACUGCA
5′-ti-Cys-GCA-10-1:
(SEQ ID NO: 51)
GGGGGUAUAGCUCAGGGGUAGAGCAUUUGACUGCAG
5′-ti-Cys-GCA-10-1:
(SEQ ID NO: 52)
GGGGGUAUAGCUCAGGGGUAGAGCAUUUGACUGCAGA

In embodiments, 5′-tiRNAs include those having a certain percent identity (e.g., 70%, 75%, 80%, 85%, 90% or even 91%, 92%, 93%, 94%, 95%, 96%, 97%, 98%, 99%, or 100% sequence identity) to any of the aforementioned 5′-Pro-tiRNAs or 5′-Cys-tiRNAs or to other 5′-tiRNAs described herein (e.g., the table above listing various human 5′-tiRNA-Pro and 5′-tiRNA-Cys tiRNAs).

As used herein, the term “percent identity” or “sequence identity” refers to percent (%) sequence identity with respect to a reference polynucleotide sequence following alignment by standard techniques. Alignment for purposes of determining percent nucleic acid sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul et al. (1990) J. Mol. Biol., 215:403-410). As an illustration, the percent sequence identity of a given nucleic acid sequence, A, to, with, or against a given nucleic acid sequence, B, (which can alternatively be phrased as a given nucleic acid sequence, A that has a certain percent sequence identity to, with, or against a given nucleic acid sequence, B) is calculated as follows:

100 multiplied by (the fraction X/Y)

where X is the number of nucleotides scored as identical matches by a sequence alignment program (e.g., BLAST) in that program's alignment of A and B, and where Y is the total number of nucleotides in B.

In yet other embodiments, the 5′-tiRNA is a heterologous nucleic acid molecule. Such a heterologous nucleic acid molecule or sequence is a nucleic acid molecule or sequence that (a) is not native to a cell in which it is introduced or (b) has been altered or mutated by the hand of man relative to its native state, or (c) has altered expression as compared to its native expression levels under similar conditions.

5′-tiRNAs and Modified 5′-tiRNAs

It is contemplated that for any of the 5′-tiRNA molecules disclosed herein, such molecules may be used in the methods disclosed herein either alone or in a modified form. Typically, modifications to the 5′-tiRNA are introduced to optimize the molecule's efficacy or biophysical properties (e.g., increasing serum stability or circulating half-life, increasing thermal stability, enhancing transmembrane delivery, reduce immunogenicity of the 5′-tiRNA, and/or targeting to a particular location or cell type).

Such modification is achieved by systematically adding or removing linked nucleosides to generate longer or shorter sequences. Further 5′-tiRNA modifications include the incorporation of, for example, one or more alternative nucleosides, alternative 2′ sugar moieties, and/or alternative internucleoside linkages.

Nucleoside Modifications

Modification of the 5′-tiRNA molecules described herein include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The 5′-tiRNA molecules may also include nucleobases in which the purine or pyrimidine base is replaced with other heterocycles, for example 7-deaza-adenine, 7-deazaguanosine, 2-aminopyridine, and/or 2-pyridone. Further modification of the 5′-tiRNA molecules described herein may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

Sugar Modifications

Modifications of the 5′-tiRNA molecules described herein may also include one or more of the following 2′ sugar modifications: 2′-O-methyl (2′-O-Me), 2′-methoxyethoxy (2′-O—CH2CH2OCH3, also known as 2′-O-(2-methoxyethyl) or 2′-MOE), 2′-dimethylaminooxyethoxy, i.e., a O(CH2)2ON(CH3)2 group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH2OCH2N(CH3)2. Other possible 2′-modifications that can modify the 5′-tiRNA molecules described herein include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH2CH2CH2NH2), allyl (—CH2-CH═CH2), —O-allyl (—O—CH2-CH═CH2) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

Internucleoside Linkage Modifications

Modifications of the 5′-tiRNA molecules described herein may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### Transfection

Any of the aforementioned 5′-tiRNAs may be transfected, according to standard methods, into a cell in vivo, in vitro, or ex vivo. The 5′-tiRNA may be transfected, for example, into an induced pluripotent stem cell (iPSC), a hematopoietic stem and progenitor cell (HSPC) (Exemplary HSPCs include an iPSC-derived HSPC, HPSCs from a donor, a myeloid progenitor cell, a lymphoid progenitor cell, or a granulocyte-macrophage progenitor (GMP)). Such cells may have an autologous or banked origin. In some embodiments, transfection of one or more 5′-tiRNA molecules may be mediated by a liposome, an exosome, or a lipid nanoparticle (LNP). In some embodiments, transfection of one or more 5′-tiRNA molecules may modulate the cellular differentiation pathway of a stem-progenitor cell (SPC), iPSC, iPSC-derived HSPC, HSPC, a myeloid progenitor cell, lymphoid progenitor cell, or a GMP. Any cell transfected with one or more of the 5′-tiRNA molecules is referred herein as an engineered cell and typically includes a heterologous 5′-tiRNA.

### Transplantation

Any of the transfected cells described herein (e.g., an engineered cell), such as an iPSC-derived HPSC, a HSPC, a myeloid progenitor cell, a lymphoid progenitor cell, or a GMP, may be transplanted into a subject (e.g., a human). Cells to be transplanted may have an autologous or allogenic origin. In some embodiments, transplantation of an iPSC-derived HPSC, a HSPC, a myeloid progenitor cell, a lymphoid progenitor cell, or a GMP that is transfected with a 5′-tiRNA may be used to treat radiation therapy, chemical injury, or genotoxic injury (e.g., to the bone marrow), or to increase reconstitution of a subject's immune system after a stem cell transplant.

### Compostions

The 5′-tiRNA molecules (e.g., in an unmodified or modified form) described herein may be formulated into various compositions (including a pharmaceutical composition) for administration to a subject in a biologically compatible form suitable for administration in vivo. For example, the 5′-tiRNA molecules described herein may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22nd ed. and in The United States Pharmacopeial Convention, The National Formulary, United States Pharmacopeial, 2015, USP 38 NF 33).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates and mammals.

The 5′-tiRNAs and pharmaceutical compositions described herein may contain at least one 5′-tiRNA molecule (e.g., a 5′-5′-tiRNA-Pro-CGG-1-1 or a 5′-5′-tiRNA-Cys-GCA-10-1). As a non-limiting example, the formulations may further contain more than one 5′-tiRNA molecules (e.g., a 5′-5′-tiRNA-Pro-CGG-1-1 and a 5′-5′-tiRNA-Cys-GCA-10-1). The 5′-tiRNAs and pharmaceutical compositions described herein may be loaded into a carrier such as an exosome, liposome, or a lipid nanoparticle (LNP) according to standard methods known in the art. Such exemplary carriers are now described.

Exosomes

In one embodiment, pharmaceutical compositions of 5′-tiRNAs include exosomes. Exosomes produced from cells can be collected from the culture medium by any suitable method. Typically, a preparation of exosomes can be prepared from cell culture or tissue supernatant by centrifugation, filtration or combinations of these methods. For example, using standard methods, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 micrometer filter), gradient ultracentrifugation (for example, with sucrose gradient) or a combination of these methods. Exosomes are loaded with exogenous 5′-tiRNAs, according to standard methods, for systemic delivery to a subject (e.g., a human patient).

Liposomes

In one embodiment, pharmaceutical compositions of 5′-tiRNAs include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations. Liposomes are loaded with exogenous 5′-tiRNAs, according to standard methods, for systemic delivery to a subject (e.g., a human patient).

Lipid Nanoparticles (LNPs)

In one embodiment, pharmaceutical compositions of 5′-tiRNAs include lipid nanoparticles (LNPs). For example, the 5′-tiRNA, such as a 5′-5′-tiRNA-Pro-CGG-1-1 and/or a 5′-5′-tiRNA-Cys-GCA-10-1, may be formulated in a lipid nanoparticle such as those described in International Publication No. WO2012170930, herein incorporated by reference in its entirety. As a non-limiting example, LNP formulations may contain cationic lipids, distearoylphosphatidylcholine (DSPC), cholesterol, polyethylene glycol (PEG), R-3-[(ω-methoxy poly(ethylene glycol)2000)carbamoyl)]-1,2-dimyristyloxl-propyl-3-amine (PEG-c-DOMG), distearoyl-rac-glycerol (DSG) and/or dimethylaminobutanoate (DMA). As a non-limiting example, 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypoly ethylene glycol) or PEG-DPG (1,2-Dipalmitoyl-sn-glycerol, methoxypolyethylene glycol). The cationic lipid may be selected from any lipid known in the art such as, but not limited to, (6Z,9Z,28Z,31Z)-heptatriacont-6,9,28,31-tetraene-19-yl 4-(dimethylamino)butanoate (DLin-MC3-DMA), 1,2-dilinoleyloxy-n,n-dimethyl-3-aminopropane (DLin-DMA), C 12-200, and N,N-dimethyl-2,2-di-(9Z,12Z)-9,12-octadecadien-1-yl-1,3-dioxolane-4-ethanamine (DLin-KC2-DMA). LNPs are loaded with exogenous 5′-tiRNAs, according to standard methods, for systemic delivery to a subject (e.g., a human patient).

### Administration

The 5′-tiRNAs described herein may be administered in unmodified or modified form and such forms may, if desired, be formulated into a composition (e.g., a pharmaceutical composition including an exosome, a liposome, or a nanoparticle) for administration to a subject in a biologically compatible form suitable for administration in vitro, in vivo, or ex vivo. In general, a suitable daily dose of one or more of the 5′-tiRNAs described herein will be an amount which is the lowest dose effective to produce a therapeutic effect. The 5′-tiRNAs described herein may be administered by injection, e.g., intravenous, intramuscular, intraperitoneal, or subcutaneous. For example, the 5′-tiRNAs described herein can be systemically administered to a subject via intravenous injection. Alternatively, the 5′-tiRNAs described herein may be administered by injection transfection, such as transfection of in vitro or ex vivo cells.

### Treatment

Any of the aforementioned 5′-tiRNA molecules, engineered cells (e.g., cells transfected with a 5′-tiRNA), or compositions (e.g., pharmaceutical compositions) can be used for the treatment of a subject (e.g., a human) with a disease, a disorder, a trauma, a chemical injury, a radiation injury, a genotoxic injury, or is recovering from a post-surgical procedure, or has received a stem cell transplant. In some embodiments, the disease or disorder is a microbial infection, such as a fungal infection (e.g., Candida albicans). In some embodiments, the disease or disorder is a bacterial infection. In some embodiments, the disease or disorder is a viral infection. In some embodiments, the disease or disorder is a blood disorder such as sepsis or septicemia. In some embodiments, the trauma is, for example, a bodily injury, a wound, a bone fracture, a traumatic brain injury, etc. In some embodiments, the chemical injury, radiation injury, or genotoxic injury is to bone marrow. In some embodiments, the treatment following a stem cell transplant (e.g., an autologous or allogenic transplant) is to increase reconstitution of the subject's immune system. Treatment with the 5′-tiRNA molecules, engineered cells (e.g., cells transfected with a 5′-tiRNA), or compositions (e.g., pharmaceutical compositions) described herein can be used for any disease or disorder which is mitigated by an augmentation of hematopoiesis in the subject in need thereof. An augmentation of hematopoiesis can increase the number of myeloid progenitor cells, neutrophils, granulocytes, or macrophages in the subject.

### Examples

As is explained in detail below, we have identified processed tRNAs (tiRNA) which alter HSPC proliferation. The findings below indicate that specific stromal cells transfer a species of tiRNA directly to hematopoietic cells through extracellular vesicles (EVs), creating a cell communication schema distinct from the ligand-receptor paradigm. This signaling process is one that is increased under physiologic stress and represents a distinctive, perhaps ancient form of niche regulation.

By using RNA reporter animal models and RNA-sequencing, we have demonstrated that osteoblast-derived EVs are enriched in tiRNAs. One tiRNA in particular, 5′-ti-Pro-CGG-1, induced an increase in protein translation and cell cycle and enhanced differentiation of transfected mouse granulocyte macrophage progenitor cells (GMPs), as assessed by cell surface markers, functional phagocytosis, and killing assays. Notably, upregulating EV-mediated transfer of tiRNAs (e.g., 5′-ti-Pro-CGG-1) improved the animal's hematopoietic recovery from genotoxic injury and improved their survival after septic challenge.

Moreover, we have demonstrated that osteoblastic cells in the bone marrow (BM) niche elaborate extracellular vesicles that are taken up by hematopoietic progenitor cells in vivo. Genotoxic or infectious stress rapidly increased stromal-derived extracellular vesicle transfer to granulocyte-monocyte progenitors. The extracellular vesicles contained processed tRNAs (tiRNAs) which modulate protein translation. 5′-ti-Pro-CGG-1 was preferentially abundant in osteoblast-derived extracellular vesicles and, when transferred to granulocyte-monocyte progenitors (GMP), increased protein translation, cell proliferation, and myeloid differentiation. Upregulating EV transfer improved hematopoietic recovery from genotoxic injury and survival from fungal sepsis. Therefore, EV-mediated tiRNA transfer provides a stress-modulated signaling axis in the BM niche distinct from conventional cytokine-driven stress responses.

The following are representative examples of the invention and should not be construed as limiting.

EVs Shuttle Proteins and RNA from Osteoblastic to Hematopoietic Cells in the BM

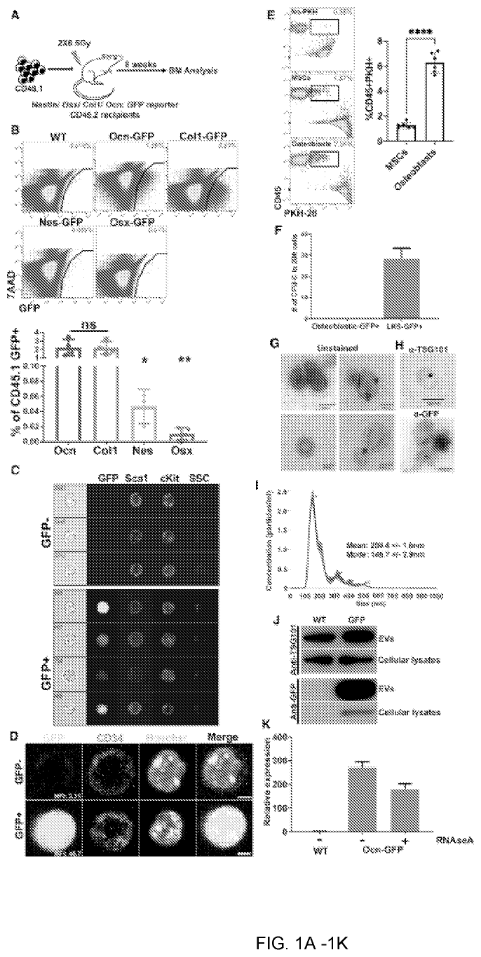

Exchange of cellular material between BM mesenchymal stroma (BMMS) and HSPCs was evaluated using mouse reporter models with GFP or GFP^{Topaz} expressed under control of promoters active in specific mesenchymal cells that are known extrinsic regulators of HSPCs (FIG. 1A; Morrison and Scadden, 2014). Osteocalcin GFP-Topaz (Ocn-GFP^{Topaz}) (Bilic-Curcic et al., 2005) and collagen 1-GFP (Col1-GFP) (Kalajzic et al., 2003) marked osteoblastic cells, Osterix-Cre::GFP (Osx-GFP) (Rodda and McMahon, 2006) marked osteoprogenitor cells, and nestin-GFP (Nes-GFP) (Mignone et al., 2004) marked primitive mesenchymal stromal cells (MSCs). GFP is 27 kDa, prohibiting its intercellular transfer through gap junctions (upper limit, 1 kDa; Nielsen et al., 2012).

Mice were transplanted with wild-type (WT) congenic CD45.1 BM following lethal irradiation. After 8 weeks, transplanted BM cells were assessed for the presence of GFP (FIG. 1A). CD45.1 GFP+ cells were 40-fold more abundant in Ocn-GFP^{Topaz} and Col1-GFP mice than in Nes-GFP or Osx-GFP recipients (FIGS. 1B and 7A). The frequency of GFP+ mesenchymal cells did not correlate with GFP labeling of hematopoietic cells (FIG. 7B). To rule out the effect of radiation, we demonstrated in Ocn-GFP^{Topaz} mice that GFP labeling within the hematopoietic compartment was comparable in transplanted and non-transplanted animals (FIG. 7C). Evident cytoplasmic GFP signal in single hematopoietic cells was observed by imaging flow cytometry, ruling out the possibility of osteoblasts in doublets contributing to the signal (FIGS. 1C and 7D). Confocal microscopy confirmed that GFP was cytoplasmic rather than non-specifically membrane bound (FIGS. 1D and 7D). We confirmed the enhanced production of EVs by osteoblasts in a co-culture system of PKH-26-labeled osteoblasts or MSCs with primary hematopoietic progenitors. Osteoblasts labeled 3 times more granulocyte macrophage progenitors (GMPs) compared to MSCs (FIG. 1E). Furthermore, the hematopoietic origin of the GFP+ cells was confirmed using a colony-formation assay: GFP+, Lin-ckit+Sca1+ (LKS) formed GFP− colonies in methylcellulose in contrast to the GFP+CD45− osteoblastic cells from the same animals, which did not form any colonies under hematopoietic cell culture conditions (FIGS. 1F and 7E-H).

To investigate the transfer of GFP via EVs, we performed transmission electron microscopy (TEM) analysis, which revealed cup-shaped, membrane-bound vesicles in Ocn-GFP^{Topaz} BM-derived EVs (FIG. 1G). Nanoparticle tracking analysis revealed vesicles with a mean size of 209.4 nm (±1.6) and a mode of 148.7 nm (±2.9), in keeping with exosome dimensions (FIG. 1I; Mathieu et al., 2019; van Niel et al., 2018). The exosome-specific protein, tumor susceptibility gene 101 (TSG101) was present on the EVs as confirmed by TEM (immunogold staining) and western blotting (WB) (FIGS. 1H and 1J). GFP was similarly detected in EV preparations by TEM and WB at the protein level (FIGS. 1H and 1J). Additionally, GFP mRNA was detected by qPCR in RNaseA-treated Ocn-GFP^{Topaz} BM EVs, which was transferred to primary ex vivo cultured GMPs (FIGS. 1K and 7J). Finally, the exosome-defining tetraspanins, CD81 and CD9, were evident on the surface of BM EVsby flow cytometry (FIG. 7I). Together, these findings demonstrate that, among BMMSs, osteoblasts are producers of EVs of endocytic origin that transfer GFP protein and mRNA to hematopoietic cells in vivo.

### GMPs are the Most Abundant EV Recipients Among HSPCs

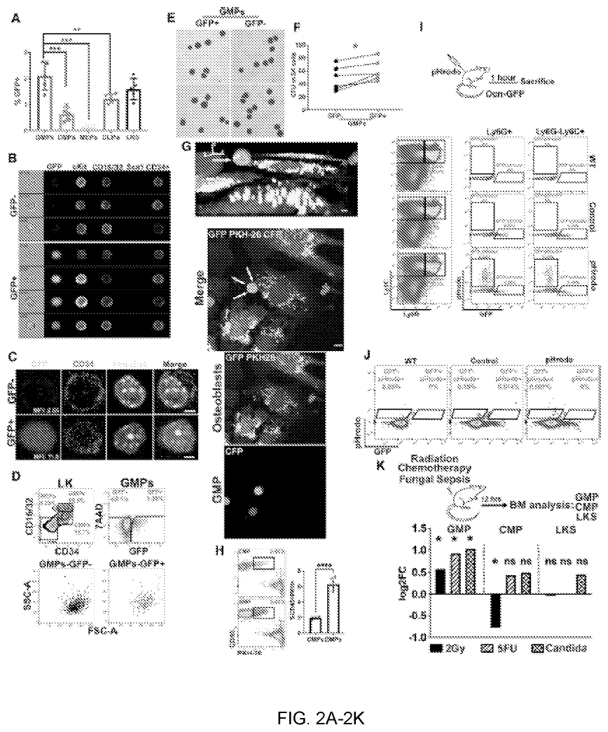

Given the role of BMMSs in the regulation of HSPC function (Kfoury and Scadden, 2015) and the experimental evidence demonstrating that alteration of specific BMMSs results in myeloid malignancies (Dong et al., 2016; Kode et al., 2014; Raaijmakers et al., 2010), we hypothesized that BMMS-derived EVs might regulate HSPCs. Using uptake of GFP as an indicator for EV uptake, we examined HSPC populations: LKS; Lin−cKit+Sca1−CD34+CD16/32lo common myeloid progenitors (CMPs); Lin−cKit+Sca1− CD34+CD16/32hi (GMPs); Lin−cKit+Sca1−CD34−CD16/32lo megakaryocyte erythroid progenitors (MEPs); and Lin-interleukin-7R (IL-7R)+cKit+Sca1+ common lymphoid progenitors (CLPs) in the BM of the Ocn-GFP^{Topaz} mice by flow cytometry. GMPs and LKS were labeled at a comparable frequency, which was significantly higher than CMPs, MEPs, and CLPs (FIGS. 2A and 8A). However, the higher frequency of GMPs (0.95%±0.15%) compared to LKS (0.28%±0.05%) in BM mononuclear cells results in very low numbers of labeled LKS and significantly higher numbers of labeled GMPs. Labeling of Lin−, cKit+, Sca1+, CD150+, and CD48− longterm HSCs (LT-HSC) was negligible (FIG. 8B). Higher level but similarly distributed EV uptake was observed using the Col1-GFP mouse model (FIGS. 8C and 8D). However, given that the Col1-GFP model labels a wider population of osteoblasts and pre-osteoblasts in addition to the specificity of the Ocn-GFP^{Topaz} to matrix-forming osteoblasts (Bilic-Curcic et al., 2005), we chose to proceed with the latter model for follow-up experiments to ensure we are analyzing a homogeneous population of EVs. Imaging flow cytometry and confocal microscopy confirmed single-cell GMP^{GFP+} cells and cytoplasmic GFP (FIGS. 2B and 2C). Scatter properties and Wright-Giemsa staining did not reveal any morphological differences between GFP+ GMPs (GMP^{GFP+}s) and GFP− GMPs (GMP^{GFP-}s) (FIGS. 2D and 2E). However, GMP^{GFP+} s were enriched in colony-forming unit capacity with comparable colony size (FIGS. 2F and 8F). Among Lin+ cells in the BM, CD11b+ myeloid cells had the highest frequency of labeling (FIGS. 8G and 8H). The transfer of EVs between osteoblasts and GMPs was confirmed by confocal imaging of a co-culture between PKH-26-labeled osteoblasts and GMPs isolated from beta actin-enhanced cyan fluorescent protein (CAG-ECFP) animals (FIGS. 2G and 8E). White arrows point toward PKH-26-labeled vesicles in GMPs transferred from osteoblasts (FIG. 2G). These vesicles were not detected in GMPs in the absence of labeled osteoblasts (FIG. 8E). Using a similar co-culture system, we confirmed the preferential uptake of osteoblast-derived EVs by GMPs compared to CMPs (FIG. 2H).

Given that GMPs give rise to phagocytic cells (Akashi et al., 2000), we tested whether GMP^{GFP+} cells simply had greater phagocytic ability by injecting Ocn-GFP^{Topaz} mice with E. coli particles labeled with a pH-sensitive dye (pHrodo) that fluoresces within the acidic milieu of the phagosome (Lenzo et al., 2016). Phagocytic (pHrodo-positive) Ly6G−Ly6C+ monocytes and Ly6G+ granulocytes were GFP− negative and hence were not labeled with EVs (FIG. 2I), while both GMP^{GFP+}s and GMP^{GFP-}s were not capable of phagocytosis (pHrodo-negative; FIG. 2J). These data in combination with the data presented in FIG. 1 argue against the uptake of free unbound GFP by phagocytosis but rather through EV-mediated transfer.

To further highlight the regulated nature of this process and rule out randomness, we tested the effect of three stress states on EV transfer to GMPs: genotoxicity from low-dose [symbol] -irradiation or 5-fluorouracil (5FU) and inflammation induced by systemic C. albicans infection. The frequency of GFP uptake was selectively increased in GMPs (1.5- to 2-fold), but not in CMPs or LKS 12 hrs post-exposure to the three stresses with no major changes in the absolute counts of GMPs (FIGS. 2K and 8I-Q). The increase in the frequency of the EV-labeled GMPs (GMP^{GFP+}) was prior to the selective changes in the absolute numbers of total GMPs at 24, but not in CMPs or LKS (FIGS. 8R-T), consistent with an increase in EV uptake rather than rapid proliferation or differential BM retention of GMPs and highlighting a distinctive effect of EVs on GMPs under stress.

tiRNAs are the Most Abundant sncRNAs in Osteoblastic EVs

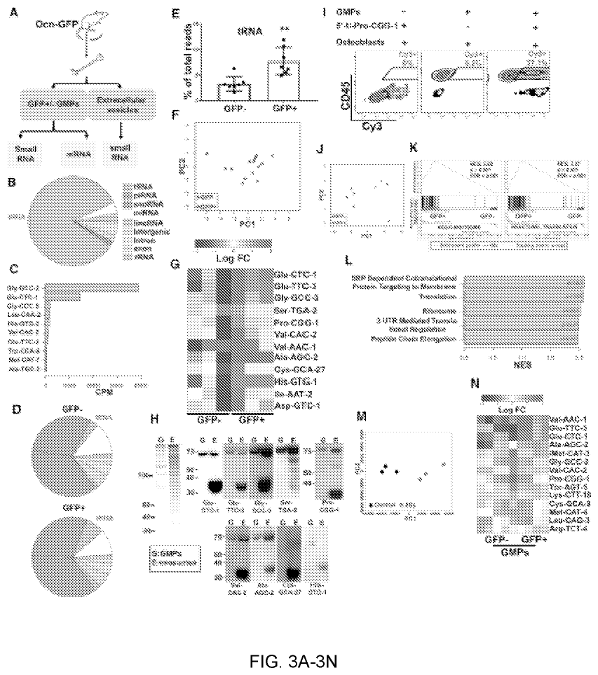

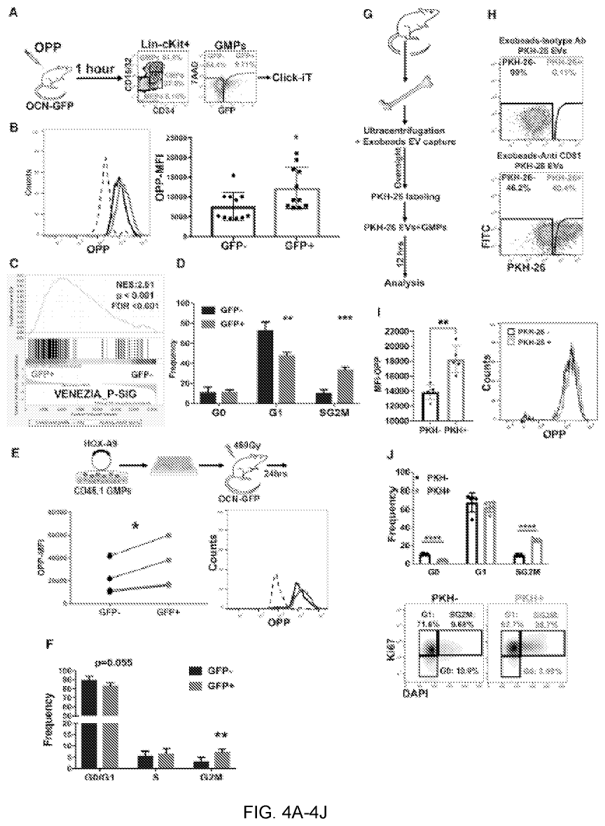

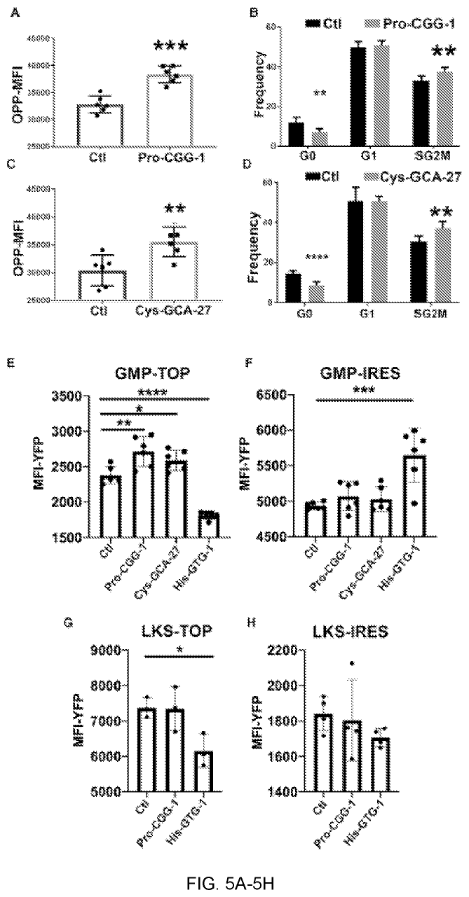

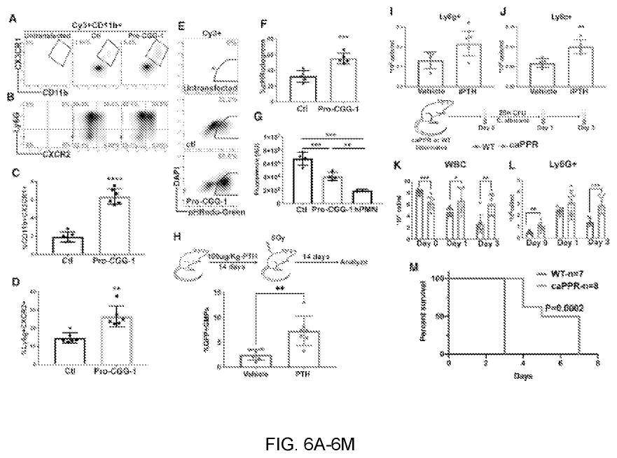

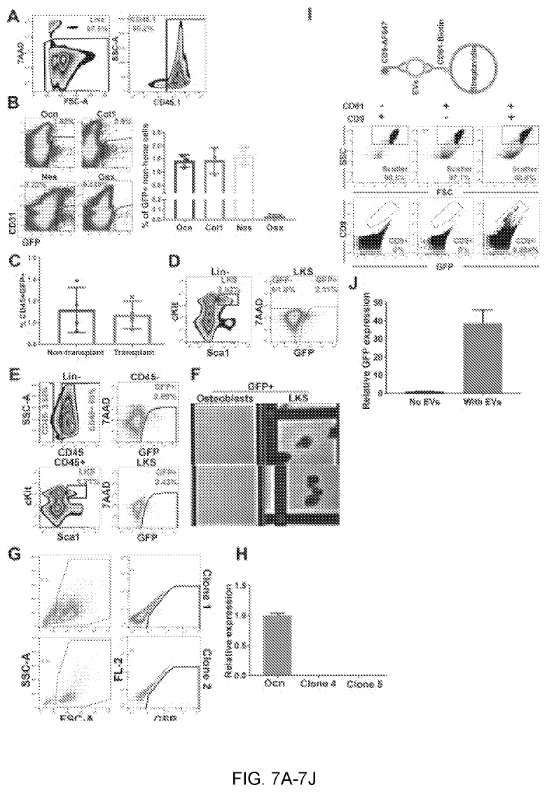

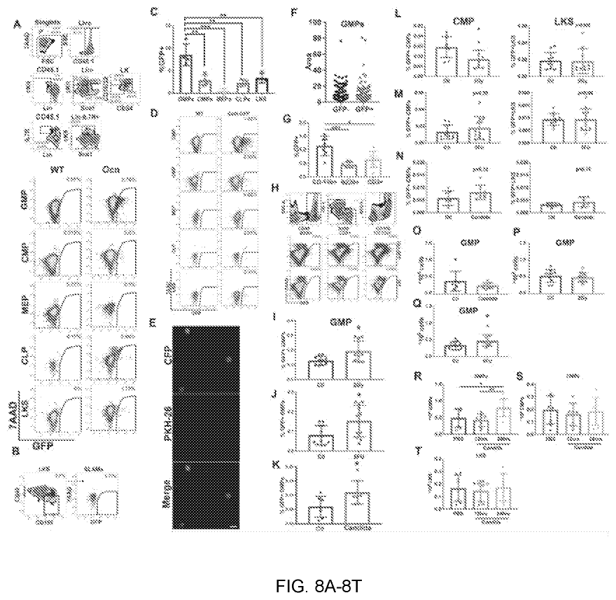

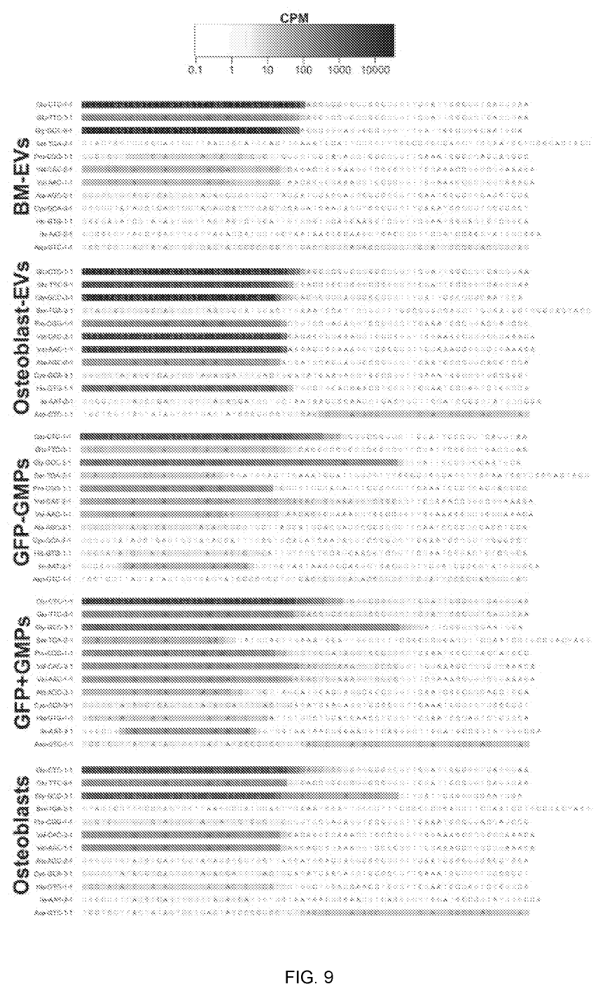

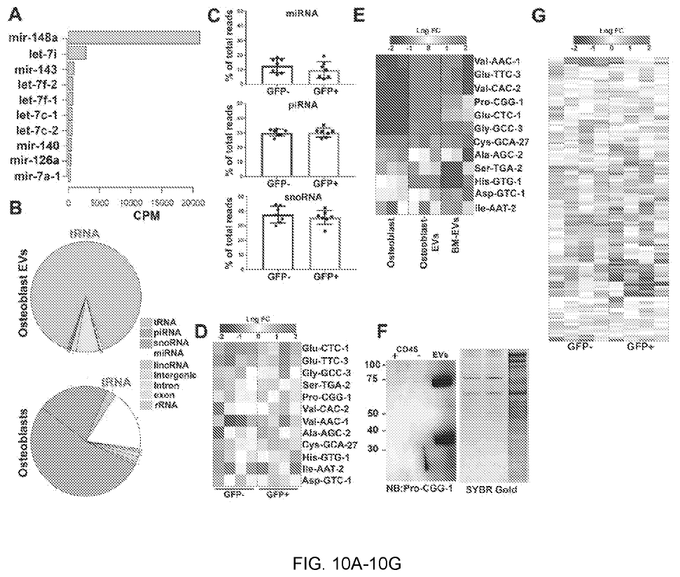

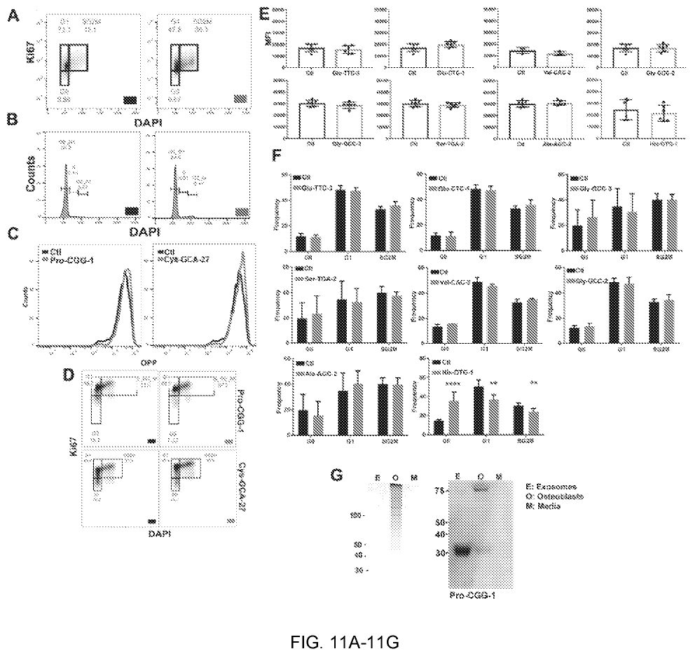

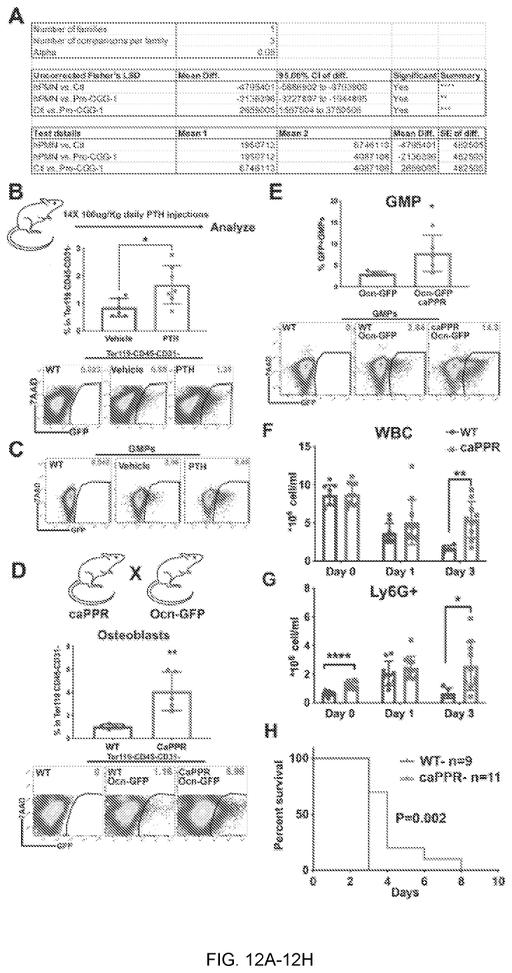

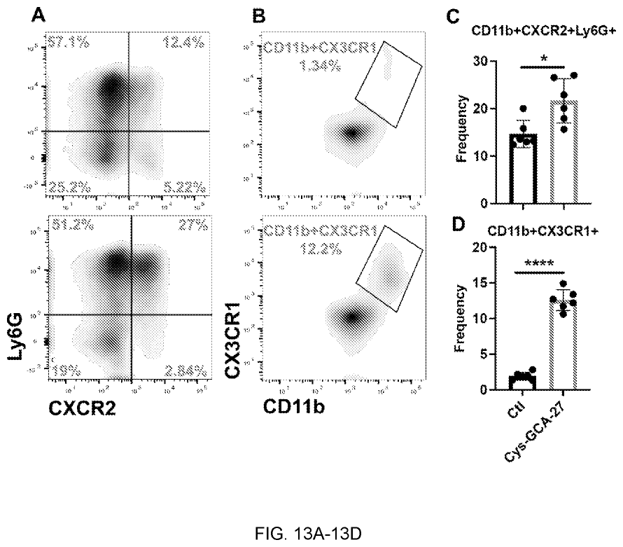

EVs carry proteins, lipids, metabolites, and nucleic acids as cargo (Keerthikumar et al., 2016). The most abundant nucleic acids in EVs are mRNAs and sncRNAs (Valadi et al., 2007; Wei et al., 2017). The sncRNA content of BM-derived EVs and of GMP^{GFP+}s and GMP^{GFP-}s from the Ocn-GFP^{Topaz} mouse model was analyzed by RNA sequencing (FIG. 3A). The vast majority (85% of reads) of EV sncRNAs consisted of tRNAs (FIG. 3B), with tRNAs coding for Gly-GCC-2, Glu-CTC-1, and Gly-CCC-5 as the most abundant (FIG. 3 and FIG. 9). Among EV miRNAs, mir-148, let-7i, and mir-143 were the most represented (FIG. 10A).

In GMP^{GFP+}s and GMP^{GFP-}s, the majority of sncRNAs were piRNAs and snoRNAs, and miRNAs were more abundant than tRNAs (FIG. 3D). This finding is similar to published reports of cultured human mesenchymal cells (Baglio et al., 2015), glioma cells (Wei et al., 2017), T cells (Chiou et al., 2018), and HEK293T (Shurtleff et al., 2017). Interestingly, the overall level of tRNAs was more than 2-fold higher in GMP^{GFP+}s compared to GMP^{GFP-}s, a distinctive finding among the sncRNAs (FIG. 3E and FIG. 10C). In addition, the GMP^{GFP+} and GMP^{GFP-} cells had distinct tRNA species levels by principal-component analysis (PCA) (FIG. 3F). Twelve tRNAs had significantly higher levels in GMP^{GFP+}s compared to GMP^{GFP-}s in two independent experiments (FIG. 3G and FIG. 10D) and were detected in BM EVs (Table S1). EVs derived from cultured primary osteoblasts were also dominated by tRNA (90% of small RNA reads) and were markedly increased compared to tRNAs in the originating osteoblasts (FIG. 10B). Val-AAC-1, Ser-TGA-2, Pro-CGG-1, Glu-TTC-3, Glu-CTC-1, and His-GTG-1 were particularly abundant in osteoblast-derived EVs (FIG. 10E).

Northern blot (NB) analysis on total RNA from BM-derived EVs confirmed the presence of seven out of the top ten differentially abundant tRNAs within EV-labeled GMPs (FIG. 3H). Interestingly, smaller tRNAs of around 35 nt were much more abundant than certain mature tRNAs within BM EVs and could not be detected in CD45+ or CD45_ BM cellular RNA (FIG. 3H and FIG. 10F). These smaller tRNAs had the size of tiRNAs, originally considered a byproduct of tRNA degradation (Borek et al., 1977; Speer et al., 1979) but increasingly recognized as a regulated tRNA-processing product modulating protein translation (Anderson and Ivanov, 2014; Fricker et al., 2019; Kim et al., 2017; Yamasaki et al., 2009). Through their effect on translation, tiRNAs enable cell tolerance of stress conditions, including oxidation, UV irradiation, heat shock, and starvation (Fricker et al., 2019; Ivanov et al., 2011; Yamasaki et al., 2009). Probes for Cys-GCA-27, His-GTG-1 detected only tiRNA (not tRNA) within EVs (FIG. 3H).

We confirmed the transfer of tiRNAs from osteoblasts to GMPs through a co-culture assay of primary GMPs and primary osteoblasts producing Cy3-labeled synthetic 5′ tiRNA Pro-CGG-1 (5′ti-Pro-CGG-1) (FIG. 3I). Together, these findings are consistent with EV transfer of select tiRNAs from osteoblastic cells to GMPs.

To investigate the effect of the transferred small RNAs on the recipient GMPs, we performed mRNA sequencing of GMP^{GFP+}s and GMP^{GFP-}s, which revealed distinctly different patterns of gene expression, as shown by PCA (FIG. 3J), with 21 significantly upregulated and 108 downregulated mRNAs (FIG. 10G). Pathway enrichment analysis using gene set enrichment analysis (GSEA) (Subramanian et al., 2005) indicated the upregulation of ribosomal and protein-translation-related genes in GMP^{GFP+} cells (FIGS. 3K and 3L). Sequencing of EV mRNA was not performed due to diminished ribosomal RNA peaks, a finding that has been reported by others (Wei et al., 2017). To investigate the effect of stress on the tRNA content of EVs and GMP^{GFP+}s, small RNA sequencing was performed on BM EVs and GMPs from Ocn-GFP animals 12 hrs post-irradiation (2 Gy). PCA and individual gene expression levels demonstrated a distinct tRNA content between EVs from control and irradiated animals (FIG. 3M). Analysis of the GMPs detected 14 tRNAs that are significantly more abundant in irradiated GMP^{GFP+}s compared to GMP^{GFP-}s from the same animals (FIG. 3N).

### Osteoblastic EVs Enhance Protein Translation and Proliferation in Recipient GMPs

To validate this upregulation of protein synthesis machinery, we performed an in vivo protein translation assay by injecting Ocn-GFP^{Topaz} mice with O-propargyl-puromycin (OPP), a molecule incorporated into nascent peptides that enables flow cytometric measurement of protein synthesis rates (Liu et al., 2012). In agreement with our pathway analyses, a significant increase in protein synthesis was observed in GMP^{GFP+} cells (FIGS. 4A and 4B). These findings have two potential explanations: (1) cells with high protein synthesis preferentially take up EVs or (2) EV uptake leads to higher protein translation. To discriminate between these, we used a model in which the expression of Homeobox-A9 (HoxA9) results in the differentiation arrest of primary mouse GMPs at a self-renewing stage, enabling clones of a uniform cell stage and phenotype to be isolated and expanded (Sykes et al., 2016). This system enables a uniform population of GMP to be adoptively transferred and the in vivo consequences of EV content transfer evaluated.

Sub-lethally irradiated Ocn-GFP^{Topaz} mice were transplanted with clonal CD45.1-HoxA9 GMP progenitors. 1 day post-transplantation, GFP was detected in the adoptively transferred cells. Further, GFP+ cells had a significantly higher rate of protein translation (by OPP analysis) compared with GFP_ cells. These data with uniform starting GMPs suggest that protein translation is directly induced by the transfer of EV contents and argue against intrinsic differences in cells leading to selective uptake (FIG. 4E).

We hypothesized that the increased rate of protein translation would correlate with an increased rate of cell cycling. Indeed, a molecular signature of proliferating hematopoietic stem cells (Venezia et al., 2004) was enriched in GMP^{GFP+}s by GSEA analysis (FIG. 4C). The GMP^{GFP+}s demonstrated an increased frequency of cells in the S/G2M phase of cell cycle (>3-fold increase), as indicated by Ki67 staining (FIGS. 4D and 11A). The GFP+ clonal HoxA9 GMPs also had increased cell cycling in vivo (FIGS. 4F and 11B). To further confirm the specificity of this phenotype to EV uptake, we isolated BM EVs by ultracentrifugation followed by anti-CD81 magnetic bead capture and added them to cultured GMPs 12 hrs before analyzing for protein translation and cell cycle. Analysis by flow cytometry confirmed the uptake of EVs captured by anti-CD81 coated beads, but not by an isotype control (FIGS. 4G and 4H). Cells labeled by the EVs demonstrated an enhanced rate of protein translation (FIG. 4I) and cellular proliferation (FIG. 4J).

Specific tiRNAs in Osteoblastic EVs Enhance Protein Translation and Cellular Proliferation

Because tiRNAs are enriched in mouse BM EVs, we tested whether the tiRNA equivalents of the top ten differentially abundant tRNAs in GMP^{GFP+} increased protein translation and cell cycling. Synthetic tiRNAs or a piRNA control sequence (5′ phosphorylated and 3′-Cy3 labeled) were transfected into primary mouse GMPs; protein translation and cell cycle were assessed 24 hrs post-transfection. 5′-ti-Pro-CGG-1 and 5′-ti-Cys-GCA-27 significantly increased the rate of protein translation in Cy3+ cells, whereas the other tiRNAs did not (FIGS. 5A, 5C, 11C, and 11E). Similarly, 5′-ti-Pro-CGG-1 and 5′-ti-Cys-GCA-27 increased the frequency of cells in the S/G2M phase of the cell cycle, whereas the other tiRNAs did not except for 5′-ti-His-GTG-1, which decreased the frequency of cells in the S/G2M phase and increased those in G0. However, given its low abundance in BM EVs, we believe its effect is minor compared to 5′-ti-Pro-CGG-1 and 5′-ti-Cys-GCA-27 that are much more abundant (FIGS. 3H, 5B, 5D, 11D, and 11F). Notably, 5′-ti-Pro-CGG-1 was present in EVs isolated from primary osteoblasts by NB, whereas the mature tRNA Pro-CGG-1 (m-Pro-CGG-1) was not. In osteoblast cellular RNA, both m-Pro-CGG-1 tRNA and 5′-ti-Pro-CGG-1 were detected; however, the tiRNA was significantly less abundant in the cells than in the EVs (FIG. 11G). In contrast, neither 5′-ti-Cys-GCA-27 nor m-Cys-GCA-27 were detected in primary osteoblast EVs (data not shown), indicating a non-osteoblastic source for the 5′-ti-Cys-GCA-27 detected in total BM EVs. These data indicate that m-Pro-CGG-1 might be processed in EVs or 5′-ti-Pro-CGG-1 is sorted into EVs and that it is the tiRNA fraction that drives changes in EV-recipient cells. Notably, Pro-CGG-1 was differentially abundant in GMP^{GFP+}s compared to GMP^{GFP-}s upon irradiation (FIG. 3N), pointing toward its potential role in response to stress.

To investigate whether the tiRNA impact on protein translation is global or restricted to specific translational regulatory elements, primary GMPs were transduced with lentiviral particles encoding a nuclear targeted yellow fluorescent protein (YFP) conjugated to either the EEF1A1 5′ terminal oligopyrimidine (TOP) motif, defined by 5-15 consecutive pyrimidine nucleotides downstream of the 7-methylguanosine cap of mRNA-mediating, cap-dependent translation (Avni et al., 1994) or the encephalomyocarditis virus (ECMV) internal ribosome entry site (IRES), which mediates cap-independent translation. Both reporters are equipped with a destabilization domain (DD) that could be stabilized by adding trimethoprim (TMP) (Han et al., 2014). The destabilization domain prevented accumulated protein from before the introduction of tiRNA, affecting the assay. In agreement with the global OPP protein translation assay, both 5′-ti-Pro-GG-1 and 5′-ti-Cys-GCA-27 enhanced cap-mediated translation as demonstrated by the TOP-H2B-YFP-DD reporter (FIG. 5E) with no change in cap-independent translation as demonstrated by the IRES-H2B-YFP-DD reporter (FIG. 5F). As a control for the assay, 5′-ti-His-GTG-1, which demonstrated a trend of inhibiting global protein translation (FIG. 11E) with a reduction in cells in the S/G2M phase of the cell cycle, demonstrated a significant reduction in cap-mediated and an increase in IRES-mediated protein translation. This was demonstrated by the YFP signal in the TOP and IRES reporters, respectively (FIGS. 5E and 5F). Interestingly, both 5′-ti-Pro-CGG-1 and 5′-ti-Cys-GCA-27 had no effect on cap-mediated or cap-independent translation in LKS (FIGS. 5G and 5H), indicating that the effect of the tiRNA has cell-specific effects in GMP. The effects on protein translation are restricted to cap-dependent mechanisms in the case of ti-Pro-CGG-1 and ti-Cys-GCA-27.

### Increased Osteoblastic EVs Enhance Response to Stress

In light of the increased osteoblast-derived EV transfer to GMPs under stress followed by GMP expansion (FIGS. 2K, 8I-K, and 8R-T), we tested whether 5′-ti-Pro-CGG-1 could affect GMP differentiation in vitro when compared to the piRNA control sequence and demonstrated an enhanced rate of differentiation by immune phenotype (increased frequency of Ly6g+CXCR2+ granulocytic and CD11b+CX3CR1+ monocytic cells) and functional phagocytosis of pHRodo-labeled E. coli and C. albicans killing in differentiated cells (FIGS. 6A-6G and 12A), in addition to the increased cell cycling and protein translation previously noted (FIGS. 5A-5D). These data provide a role for osteoblast-derived EVs and their cargo in tuning GMP stress response in a regulated manner. To investigate this in an in vivo setting and because there are no robust methods to specifically inhibit or enhance osteoblastic EV transfer in vivo, we increased the number of sender osteoblastic cells and measured the effect on EV transfer and myeloid-based immunity in vivo. This was achieved either pharmacologically using intermittent recombinant PTH (iPTH) injection (Silva et al., 2011) or genetically by using the osteoblast-specific constitutively active PTH and PTH-related peptide receptor (caPPR) mouse model under the control of the collagen 1 promoter (Calvi et al., 2001). Intermittent PTH injection increased osteoblasts and osteoblast-derived EV transfer to GMPs and enhanced myeloid cell recovery 2 weeks post-radiation injury as reflected by significantly higher neutrophils and monocytes (FIGS. 6H-6J, 12B, and 12C). Because iPTH may directly affect hematopoietic cells among many others, we used the caPPR mice, which similar to iPTH injection demonstrated increased osteoblasts as well as increased EV transfer to GMPs in mice crossed with the Ocn-GFP^{Topaz} reporter (FIGS. 12D and 12E). When challenged with a lethal dose of C. albicans, CaPPR mice demonstrated a sustained increase in myeloid cell response (FIGS. 6K, 6L, 12F, and 12G) and, notably, improved survival (FIGS. 6M and 12H).

Enhanced the Rate of GMP Differentiation by 5′-ti-Cys-GCA-27

In light of the increased osteoblast-derived EV transfer to GMPs under stress followed by GMP expansion (FIGS. 2K, 8I-K, and 8R-T), we tested whether 5′-ti-Cys-GCA-27 could affect GMP differentiation in vitro, when compared to the piRNA control sequence. In order to demonstrate an enhanced rate of differentiation, an immune phenotype assay via flow cytometry was utilized (e.g., an increased frequency of Ly6g+CXCR2+ granulocytic and CD11b+CX3CR1+ monocytic cells). Similar to that of the 5′-ti-Pro-CGG-1 phenotypic analysis (FIGS. 6A-D), 5′-ti-Cys-GCA-27 transfected GMPs also showed a significant increase in monocytic and granulocytic markers, compared to piRNA control, indicating that 5′-ti-Cys-GCA-27 can also augment GMP differentiation (FIGS. 13A-D).

These above-described results identify an unconventional mechanism through which mesenchymal cells in the BM regulate the highly dynamic myeloid component of innate immunity and identify tiRNAs as an EV cargo that can alter the physiology of recipient cells. We have demonstrated in an in vivo setting through the use of reporter mice that label specific BMMS that osteoblastic cells within the BM communicate with hematopoietic progenitors via EVs, transmitting complex information through sncRNA. We show that the process occurs in vivo and is modulated by stress. Further, we provide in vivo as well as in vitro evidence that select mesenchymal cells have a higher ability to produce and transfer EVs with preferential uptake by specific hematopoietic progenitors. The cargo of tiRNA results in vesicular signaling that alters fundamental behaviors, such as cell cycle and protein translation. Specifically, 5′-ti-Pro-CGG-1 enriched in osteoblast-derived EVs can enhance protein translation, cellular proliferation, and eventually differentiation in recipient GMPs. These phenotypic changes occur without the complex signal transmission and transcriptional regulation that are necessary downstream components of traditional ligand-receptor interactions. In this way, specific stromal cells provide a stress-regulated means of directly transferring tiRNA to activate key programs of cell physiology. By enhancing protein translation, activating cell proliferation in specific myeloid progenitor cells, this tiRNA transfer augments defense against pathogens like the Candida tested here.

We further show that this is occurring in vivo in a manner that modulates the organisms' response to physiologic stress. We demonstrate that the extent of EV transfer can be modified in vivo by altering the producer cells. This resulted in improved myeloid response and infection control.

The impact of tiRNA on protein translation that we observed was surprising. Extracellular vesicles bearing tiRNA add to the repertoire of mechanisms by which niche cells can modulate parenchymal cell responses to stress, providing a mechanism that is more direct and likely more immediate than cytokine-receptor interactions. Non-coding RNA signaling is made possible by direct exchange of cell microparticles and represents a distinctive form of stress-modulated communication between niche and parenchymal cells that affects normal and aberrant tissues and may change organismal physiology to challenges, such as infection.

The above referenced examples were obtained using the following materials and methods.

### Materials and Methods

### Materials Availability

The clonal HoxA9 cell line is available upon request.

### Data and Code Availability

RNA sequencing data have been deposited at GEO “GEO: GSE127872” and are publicly available as of the date of publication. The accession number is listed in the Key resources table below.

### Animal Models

All animal experiments were approved by the Institutional Animal Care and Use committee at Massachusetts General Hospital. Wildtype CD45.2 (C57BL/6J), congenic CD45.1 (B6.SJL-Ptprc<a>Pepc<b>/BoyJ), CAG-ECFP (B6.129(ICR)-Tg(CAG-ECFP) CK6Nagy/J) and Rosa26-YFP (Rosa-YFP, B6.129X1-Gt(ROSA)26Sortm1(EYFP)Cos/J) mice were purchased from The Jackson Laboratory. Col1-GFP (Kalajzic et al., 2003), Ocn-GFP^{Topaz} (Bilic-Curcic et al., 2005), Nes-GFP (Mignone et al., 2004), Osx-Cre::GFP (Rodda and McMahon, 2006), caPPR (Calvi et al., 2001) and Oc-Cre (Zhang et al., 2002) were previously described. Gender matched mice, 10-14 weeks of age were used in all experiments unless stated otherwise.

For total BM transplant experiments, mice received 2×(6.5Gy) doses from a cesium-137 irradiator within a 4 hours period. The day after, 1×10⁶ BM nucleated cells were transplanted via retro-orbital injection. Mice were analyzed 8 weeks post-transplantation. For the clonal cell line transplant, mice received a dose of (4.5Gy). The day after, the mice received 2×(20*10⁶) cells 8 hours apart and mice were analyzed one day after.

For genotoxic stress, mice received a dose of (2Gy or 5Gy) or one intraperitoneal injection of 150 mg/Kg 5FU. For systemic fungal infection, (C57BL/6J mice received 100K CFU and CaPPR mice received 25K of C. albicans (SC5314) in 200 ul PBS through the tail vein. Mice were analyzed 12 hrs later.

For iPTH injection, mice were given 14 daily subcutaneous injections of vehicle (10 mM citric acid, 150 nM NaCl, 0.05% Tween 80) or 100 ug/Kg body weight of Y34hPTH(1-34) amide (SVSEIQLMHNLGKHLNSMERVEWLRKKLQDVHNY.NH₂) (SEQ ID NO: 53).

### HoxA9 Clonal Cell Line

The MSCVneo-HoxA9 ecotropic retrovirus was donated by Dr. David Sykes. The vector has been previously described (Calvo et al., 2000). GMPS were sorted as described above from CD45.1 and cells were cultured in a 12 well plate pre-coated with human fibronectin (EMD Millipore) in RPMI1640 media+10% Fetal Bovine Serum (FBS), 1% Penicillin/Streptomycin, 1% L-Glutamine, 10 ng/ml SCF, 5 ng/ml IL-3, 5 ng/ml IL-6. Cells were transduced 24 hours later with MSCVneo-HoxA9 retrovirus in the presence of 8 ug/ml Polybrene. The transduction was performed by spinfection (1000 g for 60 minutes at room temperature). Following the spinfection, the cells were maintained in media described above and 24 hours later, they were selected for 4 days with G418 (Geneticin, 1 mg/ml) (Invitrogen) and later maintained in cytokine media with no selection. Two weeks post transduction, cells were sorted as single cells in 96 well plate and maintained in the cytokine supplemented media for 2 weeks. Wells containing colonies were expanded and one was used for the clonal HoxA9 cell line experiment. All through, cells were maintained in a humidified incubator at 37 C, 5% CO₂. Cell line is available upon request from investigators.

### Primary Osteoblast Culture

Primary osteoblasts were prepared as previously described with minor modifications (Panaroni et al., 2015). Tibias, femurs, hips and humeri were collected from Oc-Cre hemizygous, R26-YFP homozygous mice. BM was flushed and bones were cut into small pieces that were digested in serum free a-MEM containing 2 mg/ml Collagenase type II (Worthington, Lakewood, NJ) for 2 hours at 37 C in a shaking water bath. Bone chips were washed with serum free a-MEM and resuspended in a-MEM supplemented with 10% FBS, 50 ug/ml ascorbic acid (Sigma), 1% Penicillin/Streptomycin and 1% L-Glutamine. Cells were incubated at 37 C in a humidified 5% CO₂ incubator for one week after which the media was changed. Two weeks post seeding, the bone chips and adherent cells were trypsinized and digested at 37_C in a shaking water bath for 30 minutes in serum free a-MEM supplemented with 2 mg/ml Collagenase type II. Cells were then stained with CD31-APC (MEC13.3) and CD 45-Pacific Blue (30-F11) and GFP+ CD31− CD45− osteoblasts were sorted using BD FACS Aria II and a 100 μm nozzle.

For co-culture, sorted osteoblasts were seeded in 24 well plate (50K/well), 24 hours later, cells were transfected with 0.5 ul of 100 uM stock Cy3 labeled tiRNA using lipofectamine Stem (Invitrogen) at a 1:2 ratio. Media was changed 8 hours post transfection.

For PKH-26 (Sigma-Aldrich) labeling, osteoblasts were labeled according to manufacturer's instructions and seeded in 8 chamber borosilicate coverglass system (nunc) at 25K/chamber. One day later, media was changed to 125 ul 2% FBS a-MEM before hematopoietic progenitors were added in an equal volume of 2% FBS IMDM. Twelve hours later, the co-culture was imaged by confocal microscopy.

For EV harvest, 500K sorted osteoblast were seeded in 100 mm dishes and incubated in a humidified 5% CO₂ incubator until cells reached 80% confluency. Media was then replaced with a-MEM supplemented with 2% exosome free FBS (GIBCO), 50 ug/ml ascorbic acid, 1% Penicillin/Streptomycin, 1% L-Glutamine. Media and osteoblasts were harvested 3 days later and EVs were collected using Exoeasy kit (QIAGEN). Total RNA was extracted using miRNeasy micro (QIAGEN).

### Genotyping and QPCR

Mouse tail DNA was used for genotyping using the indicated primers:

TABLE 2

| SEQ ID | Name | Sequence |
|---|---|---|
| SEQ ID NO: 54 | R26-YFP-WT | GGAGCGGGAGAAATGGATATG |
| SEQ ID NO: 55 | R26-YFP- | AAAGTCGCTCTGAGTTGTTAT |
| | Common | |
| SEQ ID NO: 56 | R26-YFP- | AAGACCGCGAAGAGTTTGTC |
| | Mutant | |
| SEQ ID NO: 57 | Ocn-GFP^{Topaz}, | CTGGTCGAGCTGGACGG |
| | Col1-GFP and | CGACGTAAC |
| | Nes-GFP-Fwd | |
| SEQ ID NO: 58 | Ocn-GFP^{Topaz}, | ATTGATCGCGCTTCTCGTTG |
| | Col1-GFP and | GGG |
| | Nes-GFP-Rev | |
| SEQ ID NO: 59 | Osx-GFP-Fwd | CTCTTCATGAGGAGGACCCT |
| SEQ ID NO: 60 | Osx-GFP-Rev | GCCAGGCAGGTGCCTGGAC |
| | | AT |
| SEQ ID NO: 61 | Oc-Cre-Mut- | GACCAGGTTCGTTCACTCA |
| | Fwd | TGG |
| SEQ ID NO: 62 | Oc-Cre-Mut- | AGGCTAAGTGCCTTCTCT |
| | Rev | CTACAC |
| SEQ ID NO: 63 | CaPPR-Col1 | GAGTCTACATGTCTAGGG |
| | | TCTA |
| SEQ ID NO: 64 | Ca-PPR-G2 | TAGTTGGCCCACGTCCTGT |

For reverse transcription Quantitative real-time polymerase chain reaction (RT-QPCR):

RNA was extracted using the RNeasy micro kit (QIAGEN). Total RNA was then converted to cDNA using the high capacity RNA to cDNA kit (Applied Biosystems). QPCR was performed using the SYBR Green PCR MasterMix kit (Applied Biosystems) using the indicated primers:

TABLE 3

| SEQ ID | Name | Sequence |
|---|---|---|
| SEQ ID NO: 65 | GFP- | GGACGACGGC |
| | Fwd | AACTACAAGA |
| SEQ ID NO: 66 | GFP- | TTGTACTCCA |
| | Rev | GCTTGTGCCC |
| SEQ ID NO: 67 | GAPDH- | AGGTCGGTGT |
| | Fwd | GAACGGATTTG |
| SEQ ID NO: 68 | GAPDH- | TGTAGACCATG |
| | Rev | TAGTTGAGGTCA |

### Flow Cytometry Analysis and Sorting

Mice were sacrificed through CO₂ asphyxia. Whole BM mononuclear cells (MNCs) were collected by crushing of bones (tibias, femurs, hips, humeri and spine) and subjecting the cells to density gradient centrifugation (Ficoll-Paque Plus, GE Healthcare) at 400 g for 25 minutes with brakes turned off. Mononuclear cells were then stained in PBS supplemented with 2% FBS using the following antibodies: CD45−APCCy7 (30F-11), Sca1-BV421 (D7), cKit-BuV395 or APCCy7 (2B8), CD16/32−BV605 or PeCy7 (2.4G32), CD34−AF647, Pe or FITC (RAM34), IL7R-Pe (A7R34), Biotinylated lineage cocktail (CD8A (53-6.7), CD3E (145-2C11), CD45R (RA3-61B2), GR1 (RB6-8C5), CD11b (M1/70), Ter119 (Ter-119), CD4 (GK1.5) followed by Streptavidin-BV711 conjugate. Granulocyte macrophage progenitors (GMPs) were identified (Lin−cKit+CD34hiCD16/32hi) using a BD FACSARIA III. CD45.1-BV650 (A20) was used for chimerism in transplant experiments. To assess EV transfer in the mature compartment of the BM, total BM cells were stained using Ter-119-Pe (Ter-119), CD71-Pe (R17217), CD11b-AF700 (M1/70), CD3e-APC (145-2C11), CD45R-eFluor450 (RA3-6B2) 7-Aminoactinomycin D (7AAD) was used as a viability dye. At least 2×106 events were collected per sample for stem and progenitor cell analysis using a BD FACSARIA I, II or II for both analysis and sorting. Analysis was performed using the FlowJo software.

For bone analysis by flow, bones (tibias, femurs, hips, humeri and spine) were cut into small pieces after crushing and digested for one hour at 37° C. in a shaking water bath at 120 rpm. The flow through was strained over 70 μm strainer, washed and stained with antibodies for Ter119-PeCy7 9Ter119), CD45-peCy7 (30F-11), CD31-APC (MEC 13.3).

### Extracellular Vesicle Collection

For RNA extraction from EVs, mice were euthanized, and BM was flushed in PBS from tibias, femurs, hips and humeri. For the collection of cultured osteoblast EVs, 500K YFP+ osteoblasts were cultured in a-MEM supplemented with 10% FBS, 1% Penicillin/Streptomycin, 1% L-Glutamine 50 ug/ml ascorbic acid (Sigma) until cells reached 80% confluency. Media was removed and cells were washed twice with pre-warmed PBS. Fresh a-MEM supplemented with 2% exosome free FBS, 1% Penicillin/Streptomycin, 1% L-Glutamine, 50 ug/ml ascorbic acid (Sigma) was added for three days after which media was collected for EV isolation. Cells were excluded by centrifugation for 5 minutes at 500 g. EVs and RNA were then isolated from the supernatant using the Exoeasy and miRneasy (QIAGEN) according to manufacturer's instructions. For nanoparticle tracking analysis (NTA), electron microscopy and WB after cell exclusion, the supernatant was transferred into a new tube and centrifuged for 20 minutes at 20,000 g. The supernatant was then passed through a 0.22 mm low protein binding filter and subjected to ultracentrifugation at 120,000 g using the SW32Ti rotor using the Optima L90K ultra-centrifuge from Beckman coulter for 120 minutes. Pellets were washed once with PBS followed by a second round of ultracentrifugation. For culture with primary GMPs, protein quantification was performed using the DC protein assay (Biorad). 100 ug were added to 50K GMPs sorted the day before and cultured in StemSpan SFEMII supplemented with 1% L-Glutamine and Penicillin/Streptomycin with no cytokines (Stem cell technologies). Cells were cultured in a humidified incubator at 37_C and 5% CO₂ for 12 hours and then washed twice with PBS-2% FBS with 7AAD. Live cells were sorted using a BD FACS ARIA II.

### Nanoparticle Tracking Analysis (NTA)

Following PBS wash and ultracentrifugation, EV pellets were analyzed using Nanosight instrument technology (NTA 3.2 Dev Build software) (5×60 s video/sample, detection threshold: 5) for nanoparticle size.

### Confocal Microscopy

GFP+/− LKS and GMPs were sorted as described above and live cells were imaged in 8 chamber borosilicate coverglass system (nunc) coated with human plasma fibronectin (EMD Millipore) using a Leica TCS SP8 confocal microscope equipped with two photomultiplier tubes, three HyD detectors and three laser lines (405 nm blue diode, argon and white-light laser) using a 63× objective at 200 Hz and bidirectional mode. 8-bit images were acquired at 512×5212 resolution and processed by Imaris software (Bitplane). For co-culture, 25*103 PKH-26 labeled primary osteoblasts/were cultured in 8 chamber borosilicate coverglass system (nunc). Sorted GMPs from Actin-CFP mice were co-cultured overnight before imaging.

### EVs Exobead Capture and PKH-26 Labeling

Extracellular vesicles were prepared by ultracentrifugation as described above and washed once with PBS. EVs were then pulled down by incubating with anti CD81-Biotin (Eat-2, Biolegend) coated streptavidin beads overnight rotating at 4 C (Exosome-Streptavidin Isolation/Detection reagent, Invitrogen). Beads were then collected using a magnet and washed 3 times with PBS supplemented with 0.1% BSA. For fluorescent labeling, pulled down EV/Bead complexes were stained using anti CD9-AF647 (MZ3-Biolegend) and analyzed using BDFACS ARIA II. For PKH-26 (Sigma-Aldrich) labeling, 200 ug of ultracentrifugation enriched EVs were pulled down using anti-CD81 coated Exobeads as described above. Captured EVs were labeled in 200 ul volume for five minutes. Labeling was stopped using an equal volume of PBS with 1% BSA and samples were washed three times according to manufacturer's instructions. The equivalent of 100 ug starting material of Exobead captured EVs labeled with PKH-26 were added to 50K sorted GMPs in StemSpan supplemented with 1% Penicillin/Streptomycin and L-Glutamine without cytokines. Cells were analyzed 12 hours later for protein translation and cellular proliferation.

### Colony Forming Assay

Equal numbers of cells were sorted as described above and reconstituted in MethoCult (M3434-Stem Cell Technologies) according to manufacturer's instructions or (M3234-Stem Cell Technologies) supplemented with 2 ng/ml mIL3 and mIL6, 10 ng/ml mSCF, 1 U/ml hEPO. Recombinant cytokines were purchased from PeproTech. Colonies were manually enumerated 10 days post seeding. Colony size was measured for at least 10 colonies in each biological replicate using ImageJ.

### Cytospins and Wright Giemsa Staining

GMPGFP+ and GMPGFP− were sorted as described above and 20K cells were immobilized on slides using the cytospin for 1 minute at 1000 rpms (Thermo Scientific Shandon) and were allowed to air dry. Slides were stained in 100% Wright-Giemsa (Siemens) for 2 min, and in 20% Wright-Giemsa diluted in buffer for 12 min. Stained cells were rinsed in deionized water, and coverslips were affixed with Permount prior to microscopy.

### Imaging Flow Cytometry

GFP+/− LKS were sorted from Ocn-GFP^{Topaz} as described above and then analyzed using Amnis ImageStream, EMD Millipore).

### Isolation of DNA and RNA

DNA for genotyping was isolated from tails or cells using DNeasy blood and tissue kit (QIAGEN). RNA was isolated using the miRNeasy micro or RNeasy micro kits depending on the downstream application. All extractions were performed according to manufacturer's instructions.

### Immunoblotting

Total BM EVs or nucleated cells were lysed in NuPAGE LDS lysis buffer (Life Technologies) and proteins were quantified using the DC protein assay (Biorad). 20 ug total proteins were loaded per lane. Immunoblotting was performed using rabbit polyclonal anti-GFP (ab290-abcam) and rabbit monoclonal anti-TSG101 (EPR7130B-abcam).

### Transmission Electron Microscopy

Negative Staining:

EV suspensions were fixed in 2% paraformaldehyde and 10 ml aliquots applied onto formvar-carbon coated gold mesh grids; specimens were allowed to adsorb for 10-20 minutes. Grids were contrast-stained in droplets of chilled tylose/uranyl acetate (10-15 min) or in 2% aqueous phosphotungstic acid (30-90 sec). Preparations were allowed to air-dry prior to examining in a JEOL JEM 1011 transmission electron microscope at 80 kV. Images were collected using an AMT digital camera and imaging system with proprietary image capture software (Advanced Microscopy Techniques, Danvers, MA).

Immunogold Staining:

Following adsorption of 10 ml aliquots of EV suspensions, grid preparations were either placed immediately on drops of primary antibody anti-TSG101, Abcam (EPR7130B), or anti-GFP (ab290-abcam) in DAKO antibody diluent). In case of GFP labeling, EVs were pre-treated briefly with filtered permeabilization solution (PBS/BSA/saponin) prior to incubation in primary antibody. Incubation in primary antibody occurred for at least 1 hour at room temperature. Grids were then rinsed on droplets of PBS and incubated in goat anti-rabbit IgG gold conjugate (Ted Pella #15727, 15 nm) or (Ted Pella #15726, 10 nm) at least 1 hour at room temperature. Grids were then rinsed on droplets of PBS, then distilled water, followed by contrast-staining for 10 minutes in chilled tylose/uranyl acetate. Preparations were air-dried prior to examining in a JEOL JEM 1011 transmission electron microscope at 80 kV. Images were collected using an AMT digital camera and imaging system with proprietary image capture software (Advanced Microscopy Techniques, Danvers, MA).

mRNA and Small RNA Sequencing and Analysis

RNA-seq libraries for gene expression were constructed using Clontech SMARTer v.3 kit (Takara). Small RNA libraries were constructed using NEBNext Multiplex Small RNA Library Prep Set for Illumina (New England Biolabs). mRNA and small RNA libraries were sequenced on Illumina HiSeq2500 instrument, resulting in approximately 30 million reads and 15 million reads per sample on average, respectively.

mRNA sequencing reads were mapped with STAR aligner (Dobin et al., 2013) using the Ensembl annotation of mm10 reference genome. Read counts for each transcript were quantified by HTseq (Anders et al., 2015), followed by estimation of expression values and detection of differential expressed using edgeR (Robinson et al., 2010) after normalizing read counts and including only those genes with CPM>1 for one or more samples. Differentially expressed genes were defined based on the criteria of >2-fold change in expression value and false discovery rate (FDR)<0.001. RPKM expression values were submitted to the GSEA tool (Subramanian et al., 2005) to analyze the enrichment of functional gene categories among differentially expressed genes.

To analyze small RNA data, adaptor trimming was performed by Trimmomatic (Bolger et al., 2014) and the reads at least 18 bp long were kept for further analyses, resulting in approximately 11.9 million reads per sample on average. Sequencing reads were aligned to mm10 reference genome using BWA aligner. To quantify the expression of various RNA species, we used the Ensembi Mus_musculus GRCm38.87 (Zerbino et al., 2018) annotation of lincRNAs, miRNAs, snoRNAs, and mRNAs; the annotation of tRNAs from GtRNAdb43; and the annotation of piRNAs from piRNABank (Sai Lakshmi and Agrawal, 2008). To identify differentially expressed miRNAs and tRNAs, their expression levels were quantified by miRExpress (Wang et al., 2015) and SALMON (Patro et al., 2017) respectively, followed by calling differentially expressed RNAs using edgeR (Robinson et al., 2010).

60 out of all 471 murine tRNA sequences annotated in GtRNAdb database (Chan and Lowe, 2016) were identified as differentially expressed between GFP− and GFP+ based on the criteria of >1.5 fold change in both batches (n=3 and n=4 respectively). To assess the pattern of coverage by mapped sequencing reads for individual differentially expressed tRNAs, we used the BWA mapper with default settings (Li and Durbin, 2009) to provide exact genomic locations of mapped reads, as the exact read mapping is not provided, by design, by the SALMON method used for the quantitation of gene expression. These patterns of coverage revealed that the majority of the small RNA reads covered 5′ regions of tRNA sequences (FIG. 9). Because tRNAs with the same anticodon sequence share extremely high sequence similarity, it was challenging to distinguish between expression levels of individual tRNAs within these groups. Among differentially expressed tRNAs, the individual members of groups with the same anticodon had sequence identity above 85%, consistent with our clustering by the CD-HIT (Fu et al., 2012) tool.

Therefore, in presenting expression values and differentially expressed tRNAs, as well as in follow-up experiments, we used one individual tRNA representative per group to represent the whole group of similar tRNA species. FIG. 9 shows the density of sequencing reads over the length of tRNA sequences for these tRNA groups in all experimental conditions. One representative sequence is shown for each group.

### In Vivo Phagocytosis Assay

Ocn-GFPTopaz mice were injected intravenously with 50 mg/kg of pHrodo labeled E-coli particles (Invitrogen) and one-hour post injection mice were sacrificed, and BM MNCs were collected, stained and analyzed as described above. tiRNA transfection of GMPs GMPs were sorted as described earlier from WT (C57Bl6/J) and 50K cells were cultured in 0.5 mls of StemSpanTMSFEMII (Stem cell technologies) supplemented with 1% L-Glutamine and Penicillin/Streptomycin in addition to mouse recombinant cytokines: 10 ng/ml SCF, 100 ng/ml TPO, 5 ng/ml IL3 and IL6 (PeproTech). Cells were transfected the day after with 0.5 ul of a 100 uM stock Cy3 labeled RNA oligos using Lipofectamine Stem (Invitrogen) at a ratio of 1:2 according to manufacturer's protocol. RNA oligos were ordered from IDT with a phosphorylated 5′ end and Cy3 labeled 3′ end with the following sequences:

TABLE 4

| SEQ ID | Name | Sequence |
|---|---|---|
| SEQ ID | Pro-CGG-1 | GGCUCGUUGG |
| NO: 69 | | UCUAGGGGUA |
| | | UGAUUCUCGC |
| | | UUCG |
| SEQ ID | Cys-GCA-27 | GCGGGUAUAG |
| NO: 70 | | CUCAGGGGUA |
| | | GAAUAUUUGA |
| | | CUG |
| SEQ ID | Ala-AGC-2 | GGGGGUGUAG |
| NO: 71 | | CUCAGUGGUA |
| | | GAGCGCGUGC |
| | | UUA |
| SEQ ID | Ser-TGA-2 | GUAGUCGUGG |
| NO: 72 | | CCGAGUGGUU |
| | | AAGGCGAUGG |
| | | ACUUG |
| SEQ ID | Gly-GCC-3 | GCAUUGGUGG |
| NO: 73 | | UUCAGUGGUA |
| | | GAAUUCUCGC |
| | | CUGCC |
| SEQ ID | Glu-CTC-1 | UCCCUGGUGG |
| NO: 74 | | UCUAGUGGUU |
| | | AGGAUUCGGC |
| | | GCUCU |
| SEQ ID | Glu-TTC-3 | UCCCUGGUGG |
| NO: 75 | | UCUAGUGGCU |
| | | AGGAUUCGGC |
| | | GCUUU |
| SEQ ID | Val-CAC-2 | GUUUCCGUAG |
| NO: 76 | | UGUAGUGGUU |
| | | AUCACGUUCG |
| | | CCUCA |
| SEQ ID | His-GTG-1 | GCCGAGAUCG |
| NO: 77 | | UAUAGUGGUU |
| | | AGUACUCUGC |
| | | AUUGU |
| SEQ ID | Control | UGUGAGUCAC |
| NO: 78 | (piRNA) | GUGAGGGCAG |
| | | AAUCUGCUC |

Half media change was performed 8 hours post transfection and cells were analyzed 24 hours post transfection.

### In Vitro Protein Translation Assay

Transfected cells were counted and 75K cells were incubated in a humidified 37° C. incubator for 30 minutes in media containing 20 uM O-Propargyl Puromycin (MedChem express). Cells were stained with the fixable LIVE/DEAD™ yellow stain according to the manufacturer's protocol followed by fixation using the Fixation/Permeabilization kit (BD Biosciences). After fixation, cells were washed with PBS supplemented with 3% BSA (Sigma) and then permeabilized using 1× perm/wash buffer (BD). Cells were stained for the OPP using the Click-iT Plus Alexa Fluor 647 Picolyl azide kit (Invitrogen) according to manufacturer's protocol and analyzed using BD-FACS ARIA II.

For TOP and IRES reporter assays, primary cells were sorted and transduced with lentiviral particles for TOP-H2B-YFP-DD or IRES-H2B-YFP-DD (Han et al., 2014) at a multiplicity of infection of 10 by spinfection at 20° C. for 1 hour at 1000 g. Cells were incubated at 37° C. overnight after which half media change was performed and cells were transfected with tiRNAs as described above. Cells were treated with 10 mM TMP 12 hours before flow analysis which was 24 hours post transfection. Before analysis, cells were washed with PBS+2% FBS and resuspended in PBS+2% FBS containing DAPI for viability.

### In Vivo Protein Translation Assay

Mice were injected intraperitoneally with 50 mg/Kg OPP and sacrificed one hour later. BM MNCs were harvested as described earlier for myeloid progenitor cell surface staining. GMPGFP+ and GMPGFP− or clonal HoxA9 cells were sorted directly in the fixation buffer from the Fixation/Permeabilization kit (BD Biosciences). Cells were then washed with PBS supplemented with 3% BSA followed by the Click-iT reaction as described above. Analysis was done using BD-FACS ARIA II.

### Cell Cycle Analysis

For the tiRNA transfected GMPs, 75K cells were harvested and stained for viability using the fixable LIVE/DEAD far red stain (Invitrogen) according to manufacturer's protocol followed by fixation and permeabilization using the Fixation/Permeabilization kit (BD Biosciences). Cells were then stained overnight at 4° C. in 1× perm/wash buffer with FITC mouse Ki67 set (BD PharMingen #556026). Cells were then washed with 1× perm wash buffer and re-suspended in PBS supplemented with 1 ug/ml 40,6-diami-dino-2-phenylindole (DAPI) (Invitrogen) and 100 ug/ml RNase A (Sigma) and incubated at room temperature for 15 minutes before analyzing by flow cytometry using BD FACS Aria II. Gates were drawn based on isotype control.

For uncultured cells, GMPGFP+ and GMPGFP− or clonal HoxA9 cells were directly sorted into fixation buffer and cell cycle staining was performed as described above.

### In Vitro Differentiation Phenotypic Analysis

Primary GMPs transfected with tiRNAs as described above were analyzed 3 days post transfection. Cells were harvested and washed once with PBS-2% FBS. Cells were then blocked for 5 minutes at room temperature using anti-mouse CD16/32 Fc block (1/50) (BD Biosciences). Cells were then incubated with the staining (Ly6g-APCCy7 (1A8), CXCR2-APC (SA044G4), CD11b-AF700 (M1/70), Ly6c-BV570 (HK1.4), CX3CR1-AF400 (SA011F11), cKit-BuV395 (2B8)) mix for 30 minutes at 4° C., washed and re-suspended in PBS-2% FBS containing DAPI (Invitrogen) for viability and analyzed using BD-FACS ARIA II. Analysis for differentiated cells was performed on live Cy3+ cells gated based on non-transfected cells.

### In Vitro Phagocytosis Assay

Primary GMPs transfected with tiRNAs as described above were analyzed 3 days post transfection. 100*103 cells were incubated with pHRodo green labeled E. coli (Invitrogen) at a ratio of 1:10 (cells:bacterial particles) for one hour at 37° C. shaking. Cells were then collected, washed twice with PBS-2% FBS and re-suspended in DAPI containing buffer for viability and analyzed using BD-FACS ARIA II. Phagocytosis was assessed in live Cy3+ cells gated based on non-transfected cells. For differentiation and phagocytosis analysis, mTPO was not added to the media.

### Northern Blot

RNA was separated by size using 15% Novex TBE-Urea gels (ThermoFisher, EC6885). The RNA gel was incubated in 20 ml 0.5×TBE with 1×SYBR Gold Nucleic Acid Gel Stain (Invitrogen, S11494) for 20 minutes and imaged using alpha imager HP.

The RNA was then transferred to positively charged nylon membranes with 0.45 mm pores (Roche, 11209299001). RNA was cross-linked to the membrane using a UV Stratalinker 1800 (Stratagene). The blot was pre-hybridized with DIG Easy Hybridization Buffer (Roche, 11603558001) for 30 minutes at 40° C. and then hybridized with DIG-5′-labeled probe overnight at 40° C. in a rotation hybridization oven (Techne). Anti-sense-tiRNA DNA oligos were ordered from IDT and labeled with DIG using the DIG Oligonucleotide Tailing Kit (Roche, 03353583910). Sequences of the probes are:

TABLE 5

| SEQ ID | Name | Sequence |
|---|---|---|
| SEQ ID NO: 79 | 5′-Ala-AGC | AAGCACGCGC |
| | | TCTACCACTG |
| | | AGCTACACCC |
| | | CC |
| SEQ ID NO: 80 | 5′-Cys-GCA-27 | AGTCAAATAT |
| | | TCTACCCCTG |
| | | AGCTATACCC |
| | | GC |
| SEQ ID NO: 81 | 5′-His-GTG-1 | AATGCAGAGT |
| | | ACTAACCACT |
| | | ATACGATCTC |
| | | GGC |
| SEQ ID NO: 82 | 5′-Pro-CGG-1 | AAGCGAGAAT |
| | | CATACCCCTA |
| | | GACCAACGAG |
| | | CC |
| SEQ ID NO: 83 | 5′-Ser-TGA-2 | AGTCCATCGC |
| | | CTTAACCACT |
| | | CGGCCACGAC |
| | | TAC |
| SEQ ID NO: 84 | 5′-Val-CAC-2 | AGGCGAACGT |
| | | GATAACCACT |
| | | ACACTACGGA |
| | | AAC |
| SEQ ID NO: 85 | 5′-Glu CTC-1 | CGCCGAATCC |
| | | TAACCACTAG |
| | | ACCACCAGGG |
| | | A |
| SEQ ID NO: 86 | 5′-Glu-TTC-3 | AGCGCCGAAT |
| | | CCTAGCCACT |
| | | AGACCACCAG |
| | | GGA |
| SEQ ID NO: 87 | 5′-Gly-GCC-3 | GAGAATTCTA |
| | | CCACTGAACC |
| | | ACCCATGC |

Membranes were washed twice with 2×SSC containing 0.1% SDS at room temperature for 5 minutes, followed by one 5-minute wash with 1×SSC containing 0.1% SDS at 40° C. Next, membranes were blocked with 10 mL of 1× blocking solution diluted in 1× Maleic Acid Buffer (Roche, 115857262001) with 0.3% TWEEN 20 for 30 minutes at room temperature. One unit of Anti-Digoxigenin-AP Fab fragments (Roche, 11093274910) was added to the blocking solution and incubated for 30 minutes at room temperature. The membrane was washed twice with 1× Washing Buffer (Roche, 115857262001) for 15 minutes. Membranes were briefly equilibrated with 10 mL 1× Detection Buffer (Roche, 115857262001). To detect DIG-labeled probing, 1 mL of CPD-Star (Roche, 12041677001) diluted 1:5 with 1× Detection Buffer was applied to the membrane and exposed to autoradiography film (Amersham, 28906845) in the dark.

C. albicans Culture

Candida albicans, wild-type strain SC5314 was grown overnight from frozen stocks in yeast extract, peptone, and dextrose (YPD) medium (BD Biosciences) with 100 mg/mL ampicillin (Sigma) in an orbital shaker at 30° C. Yeast were sub-cultured to ensure early stationary phase. After pelleting and washing with cold PBS, yeast were counted using a LUNA automated cell counter and cell density adjusted in PBS to 100,000 CFUs per 200 ml. Mice were injected via lateral tail vein.

C. albicans Killing Assay

Viable Cy3+ GMPs were sorted 8 hours post transfection and cultured in a humidified incubator at 37° C. and 5% CO₂ in Stem Span SFEMII supplemented with 1% Penicillin/Streptomycin and L-Glutamine in addition to 10 ng/ml mSCF, 5 ng/ml mIL-3 and mIL6 (Peprotech). On day 3 post tiRNA transfection 50K cells were added to a 96-well clear-bottom plate with 5×104 GMPs. C. albicans was prepared as described previously and added to each well at a multiplicity of infection of five in 100 μL of complete RPMI (RPMI 1640 with 2 mM L-glutamine, 10% heat-inactivated fetal bovine serum, and 1% penicillin-streptomycin; ThermoFisher Scientific, Waltham, MA). The plate was incubated at 37° C. and 5% CO₂ for two hours to allow mammalian cell/fungal interaction. Following co-incubation, mammalian cells were lysed with 1% 4× nonidet P40 solution (10 mM Tris HCl, 150 mM sodium chloride, and 5 mM magnesium chloride, titrated to pH 7.5) and wells were supplemented with optimized yeast growth media (MOPS-RPMI; RPMI 1640 containing 2% glucose and 0.165 M MOPS, titrated to pH 7) to support C. albicans growth. Then, 10% PrestoBlue Cell Viability Reagent (ThermoFisher Scientific) was added to each well, and the plate was incubated at 37° C. with fluorescence measured every 30 minutes for 18 hours by a SpectraMax i3x plate reader (Molecular Devices, Sunnyvale, CA). Fluorescence was plotted versus time, and the time to midcurve (inflection point) was determined using GraphPad Prism 7 software (La Jolla, CA). Healthy hu-man peripheral blood neutrophils were used as a positive control. Cells were isolated using the EasySep Direct Human Neutrophil Isolation Kit (STEMCELL Technologies).

tiRNA Transfection of GMPs

Sorted GMPs (50K) were cultured in 0.5 mls of StemSpanTMSFEMII (Stem cell technologies) supplemented with 1% L-Glutamine and Penicillin/Streptomycin in addition to mouse recombinant cytokines: 10 ng/ml SCF, 100 ng/ml TPO, 5 ng/ml IL3 and IL6 (PeproTech). Cells were transfected the day after with 0.5 ul of a 100 uM stock Cy3 labeled RNA oligos using Lipofectamine Stem (Invitrogen) at a ratio of 1:2 according to manufacturer's protocol. RNA oligos were ordered from IDT with a phosphorylated 5′ end and Cy3 labeled 3′ end with the following sequences:

(SEQ ID NO: 69)
Pro-CGG-1-
GGCUCGUUGGUCUAGGGGUAUGAUUCUCGCUUCG
(SEQ ID NO: 70)
Cys-GCA-27-
GCGGGUAUAGCUCAGGGGUAGAAUAUUUGACUG
(SEQ ID NO: 78)
Control (piRNA)-
UGUGAGUCACGUGAGGGCAGAAUCUGCUC

### In Vitro Differentiation Phenotypic Analysis

Primary GMPs transfected with tiRNAs were analyzed 3 days post transfection. Cells were harvested and washed once with PBS-2% FBS. Cells were then blocked for 5 minutes at room temperature using anti-mouse CD16/32 Fc block (1/50) (BD Biosciences). Cells were then incubated with the staining (Ly6g-APCCy7 (1A8), CXCR2-APC (SA044G4), CD11b-AF700 (M1/70), Ly6c-BV570 (HK1.4), CX3CR1-AF400 (SA011F11), cKit-BuV395 (2B8)) mix for 30 minutes at 4° C., washed and re-suspended in PBS-2% FBS containing DAPI (Invitrogen) for viability and analyzed using BD-FACS ARIA II. Analysis for differentiated cells was performed on live Cy3+ cells gated based on non-transfected cells.

### Quantification and Statistical Analysis

GraphPad PRISM 7 was used to plot data and run statistical analysis. Unpaired Student's t test was used to calculate significance unless indicated otherwise. Sample sizes were based on prior similar work without the use of additional statistical estimations. All measurements were performed on independent biological replicates unless indicated otherwise.

### Resource Table

The following table summarizes sourcing for various reagents and/or resources described herein.

TABLE 6

| REAGENT OR RESOURCE | SOURCE | IDENTIFIER |
|---|---|---|
| [symbol] | | |

[symbol]

indicates data missing or illegible when filed

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

The invention accordingly includes embodiments within the following numbered paragraphs.
1. A synthetic 5′-tiRNA.
2. The 5′-tiRNA of paragraph 1, wherein the 5′-tiRNA is between 30-37 nucleotides and comprises nucleotides capable of forming a tRNA D-arm.
3. The 5′-tiRNA of paragraph 1 or paragraph 2, wherein the 5′-tiRNA is modified.
4. The 5′-tiRNA of any one of paragraphs 1-3, wherein the 5′-tiRNA comprises a non-natural or modified nucleoside or nucleotide.
5. The 5′-tiRNA of paragraphs 3 and 4, wherein the modification is chosen from 2′-O-methyl (2′-O-Me) modified nucleoside, a phosphorothioate (PS) bond between nucleosides; and a 2′-fluoro (2′-F) modified nucleoside.
6. The 5′-tiRNA of any one of paragraphs 1-5, wherein the 5′-tiRNA has sequence identity to 5′-ti-Pro-CGG-1-1: GGCUCGUUGGUCUAGGGGUAUGAUUCUCGCUUCG (SEQ ID NO: 1).
7. The 5′-tiRNA of any one of paragraphs 1-6, wherein the 5′-tiRNA is 5′-ti-Pro-CGG-1 (SEQ ID NO: 1).
8. The 5′-tiRNA of any one of paragraphs 1-5, wherein the 5′-tiRNA has sequence identity to 5′-ti-Cys-GCA-10-1: GGGGGUAUAGCUCAGGGGUAGAGCAUUUGACUG (SEQ ID NO: 2).
9. The 5′-tiRNA of any one of paragraphs 1-5 and 8, wherein the 5′-tiRNA is 5′-ti-Cys-GCA-10-1: GGGGGUAUAGCUCAGGGGUAGAGCAUUUGACUG (SEQ ID NO: 2).
10. A lipid nanoparticle comprising a 5′-tiRNA.
11. The lipid nanoparticle of paragraph 10, wherein the 5′-tiRNA is between 30-37 nucleotides and comprises nucleotides capable of forming a tRNA D-arm.
12. The lipid nanoparticle of paragraph 10 or paragraph 11, wherein the 5′-tiRNA is modified.
13. The lipid nanoparticle of any one of paragraphs 10-12, wherein the 5′-tiRNA comprises a non-natural or modified nucleoside or nucleotide.
14. The lipid nanoparticle of paragraphs 12 and 13, wherein the modification is chosen from 2′-O-methyl (2′-O-Me) modified nucleoside, a phosphorothioate (PS) bond between nucleosides; and a 2′-fluoro (2′-F) modified nucleoside.
15. The lipid nanoparticle of any one of paragraphs 10-14, wherein the 5′-tiRNA has sequence identity to 5′-ti-Pro-CGG-1-1: GGCUCGUUGGUCUAGGGGUAUGAUUCUCGCUUCG (SEQ ID NO: 1).
16. The lipid nanoparticle of any one of paragraphs 10-15, wherein the 5′-tiRNA is 5′-ti-Pro-CGG-1 (SEQ ID NO: 1).
17. The lipid nanoparticle of any one of paragraphs 10-14, wherein the 5′-tiRNA has sequence identity to 5′-ti-Cys-GCA-10-1: GGGGGUAUAGCUCAGGGGUAGAGCAUUUGACUG (SEQ ID NO: 2).
18. The lipid nanoparticle of any one of paragraphs 10-14 and 17, wherein the 5′-tiRNA is 5′-ti-Cys-GCA-10-1: GGGGGUAUAGCUCAGGGGUAGAGCAUUUGACUG (SEQ ID NO: 2).
19. The lipid nanoparticle of any one of paragraphs 10-14, comprising two or more 5′-tiRNAs.
20. The lipid nanoparticle of paragraph 19, comprising two 5′-tiRNAs, wherein the first 5′-tiRNA comprises sequence identity to 5′-ti-Pro-CGG-1-1: GGCUCGUUGGUCUAGGGGUAUGAUUCUCGCUUCG (SEQ ID NO: 1) and the second 5′-tiRNA comprises sequence identity to 5′-ti-Cys-GCA-10-1: GGGGGUAUAGCUCAGGGGUAGAGCAUUUGACUG (SEQ ID NO: 2).
21. The lipid nanoparticle of any one of paragraphs 19 or 20, wherein the two 5′-tiRNAs are 5′-ti-Pro-CGG-1-1: GGCUCGUUGGUCUAGGGGUAUGAUUCUCGCUUCG (SEQ ID NO: 1) and 5′-ti-Cys-GCA-10-1: GGGGGUAUAGCUCAGGGGUAGAGCAUUUGACUG (SEQ ID NO: 2).
22. An engineered cell comprising any of the 5′-tiRNAs of paragraphs 1-9.
23. The cell of paragraph 22, wherein the 5′-tiRNA has sequence identity to 5′-ti-Pro-CGG-1-1: GGCUCGUUGGUCUAGGGGUAUGAUUCUCGCUUCG (SEQ ID NO: 1).
24. The cell of paragraph 23, wherein the 5′-tiRNA is 5′-ti-Pro-CGG-1-1: GGCUCGUUGGUCUAGGGGUAUGAUUCUCGCUUCG (SEQ ID NO: 1).
25. The cell of paragraph 22, wherein the 5′-tiRNA has sequence identity to 5′-ti-Cys-GCA-10-1: GGGGGUAUAGCUCAGGGGUAGAGCAUUUGACUG (SEQ ID NO: 2).
26. The cell of paragraph 25, wherein the 5′-tiRNA is 5′-ti-Cys-GCA-10-1: GGGGGUAUAGCUCAGGGGUAGAGCAUUUGACUG (SEQ ID NO: 2).
27. The cell of any one of paragraphs 22-26, comprising two or more 5′-tiRNAs.
28. The cell of paragraph 27, comprising two 5′-tiRNAs, wherein the first 5′-tiRNA comprises sequence identity to 5′-ti-Pro-CGG-1-1: GGCUCGUUGGUCUAGGGGUAUGAUUCUCGCUUCG (SEQ ID NO: 1) and the second 5′-tiRNA comprises sequence identity to 5′-ti-Cys-GCA-10-1: GGGGGUAUAGCUCAGGGGUAGAGCAUUUGACUG (SEQ ID NO: 2).
29. The cell of paragraph 28, wherein the two 5′-tiRNAs are 5′-ti-Pro-CGG-1-1: GGCUCGUUGGUCUAGGGGUAUGAUUCUCGCUUCG (SEQ ID NO: 1) and 5′-ti-Cys-GCA-10-1: GGGGGUAUAGCUCAGGGGUAGAGCAUUUGACUG (SEQ ID NO: 2).
30. The cell of any one of paragraphs 22-29, wherein the cell is an induced pluripotent stem cells (iPSC)-derived hematopoietic stem and progenitor cells (HSPC), a HSPC, a myeloid progenitor cell, or a granulocyte-macrophage progenitor (GMP).
31. The cell of paragraph 30, wherein the HSPC is a lymphoid progenitor cell.
32. The cells of any one of paragraphs 22-31, wherein the cell is autologous.
33. The cells of any one of paragraphs 22-32, wherein the cell is banked.
34. A treatment method comprising the step of: transfecting a cell, in a subject, with any of the 5′-tiRNAs of paragraphs 1-9 or contacting a cell, in a subject, with the lipid nanoparticles of paragraphs 10-21 under conditions effective to treat the subject.
35. A treatment method comprising the step of: transplanting any one of the cells of paragraph 30 or paragraph 31 into a subject under conditions effective to treat a subject.
36. The method of paragraphs 34 or 35, wherein the method treats a disease or disorder.
37. The method of paragraph 36, wherein the disease or disorder is a microbial infection.
38. The method of paragraph 37, wherein the microbial infection is a fungal infection.
39. The method of paragraph 38, wherein the fungus is Candida.
40. The method of paragraph 37, wherein the microbial infection is a bacterial infection.
41. The method of paragraph 36, wherein the disease or disorder is sepsis.
42. The method of paragraph 36, wherein the treatment increases the number of neutrophils, granulocytes or macrophages in the subject.
43. The method of paragraph 36, wherein the treatment increases myeloid cell production in vivo.
44. The method of paragraph 36, wherein the treatment is post-surgically administered.
45. The method of paragraph 36, wherein the treatment is administered to treat a trauma.
46. The method of paragraph 36, wherein the treatment increases reconstitution of recovery after a stem cell transplant, after radiation therapy, or after a chemical injury to bone marrow.
47. The method of paragraph 46, wherein the transplant is autologous.
48. The method of paragraph 46, wherein the transplant is allogenic.
49. A composition comprising any one of the 5′-tiRNAs of paragraphs 1-9.
50. The composition of paragraph 49, wherein the 5′-tiRNAs are formulated in a liposome, an exosome, or a lipid nanoparticle.
51. The composition of paragraph 49, comprising the engineered cells of any one of paragraphs 22-33.
52. The composition of any one of paragraphs 49-51, wherein the composition is a pharmaceutical composition.
53. A method of administering a 5′-tiRNA to a subject to treat a disease or disorder, the method comprising: administering to the subject a therapeutically effective amount of the composition of any one of paragraphs 49-52.
54. The method of paragraph 53, wherein the disease or disorder is a microbial infection.
55. The method of paragraph 54, wherein the microbial infection is a fungal infection.
56. The method of paragraph 55, wherein the fungus is Candida.
57. The method of paragraph 54, wherein the microbial infection is a bacterial infection.
58. The method of paragraph 53, wherein the disease or disorder is sepsis.
59. The method of paragraph 53, wherein the treatment increases the number of neutrophils, granulocytes or macrophages in the subject.
60. The method of paragraph 53, wherein the treatment increases myeloid cell production in vivo.
61. The method of paragraph 53, wherein the treatment is post-surgically administered.
62. The method of paragraph 53, wherein the treatment is administered to treat a trauma.
63. The method of paragraph 53, wherein the treatment increases reconstitution of recovery after a stem cell transplant, after radiation therapy, or after a chemical injury to bone marrow.
64. The method of paragraph 63, wherein the transplant is autologous.
65. The method of paragraph 63, wherein the transplant is allogenic.
66. A method of increasing myeloid cell production in a subject, the method comprising: administering to the subject a therapeutically effective amount of the composition of any one of paragraphs 49-52.
67. A method for modulating the differentiation of a stem-progenitor cell (SPC), comprising transfecting a stem-progenitor cell with one or more 5′-tiRNAs of any one of paragraphs 1-9.
68. The method of paragraph 67, wherein the stem-progenitor cells are induced pluripotent stem cells (iPSC).
69. The method of paragraph 67 wherein the stem-progenitor are hematopoietic stem-progenitor cells (HSPC).
70. The method of paragraph 67, wherein the stem-progenitor cells are granulocyte-macrophage progenitor cells (GMP).
71. The method of paragraph 67, wherein the stem-progenitor cells are isolated from a subject.
72. The method of paragraph 67, wherein the stem-progenitor cells are peripheral blood stem-progenitor cells.
73. The method of paragraph 67, wherein the 5′-tiRNA is formulated in an exosome, a liposome, or a lipid nanoparticle.
74. A method of delivering a 5′-tiRNA to an induced pluripotent stem cell (iPSC) or an iPSC population, the method comprising:
a. transfecting the iPSC or the iPSC population with a 5′-tiRNA of any one of paragraphs 1-9 in vitro; and
b. optionally, culturing the iPSC or the iPSC population in vitro;
thereby delivering the 5′-tiRNA to the iPSC or the iPSC population.
   75. The method of paragraph 74, further comprising culturing the transfected iPSC or the iPSC population.
   76. The method of paragraph 74, wherein the iPSC or the iPSC population is autologous.
   77. The method of paragraph 74, wherein the iPSC or the iPSC population is banked.
   78. A method of delivering a 5′-tiRNA to a hematopoietic stem and/or progenitor cell (HSPC) or an HSPC population, the method comprising:
a. transfecting the HSPC or the HSPC population with a 5′-tiRNA of ay one of paragraphs 1-9 in vitro; and
b. optionally, culturing the HSPC or the HSPC population in vitro;
thereby delivering the 5′-tiRNA to the HSPC or the HSPC population.
   79. The method of paragraph 78, wherein the HSPC is a hematopoietic stem cell (HSC).
   80. The method of any one of paragraphs 74-79, wherein the iPSC, iPSC population, HSPC, or HSPC population is a human cell or sample.
   81. An iPSC or iPSC population transfected with a 5′-tiRNA of any one of paragraphs 1-9.
   82. The iPSC or iPSC population of paragraph 81, comprising differentiating the iPSC or iPSC population.
   83. The iPSC or iPSC population of paragraphs 81-82, wherein the iPSC or iPSC population is autologous with respect to a patient to be administered the cell.
   84. The iPSC or iPSC population of paragraphs 81-82, wherein the iPSC or iPSC population is allogenic with respect to a patient to be administered the cell.
   85. An HSPC or HSPC population transfected with a 5′-tiRNA of any one of paragraphs 1-9.
   86. The HSPC or HSPC population of paragraph 85, comprising differentiating the HSPC or HSPC population.
   87. The HSPC or HSPC population of paragraphs 85-86, wherein the HSPC or HSPC population is autologous with respect to a patient to be administered the cell.
   88. The HSPC or HSPC population of paragraphs 85-86, wherein the HSPC or HSPC population is allogenic with respect to a patient to be administered the cell.
   89. An GMP transfected with a 5′-tiRNA of any one of paragraphs 1-9.
   90. The GMP of paragraph 89, comprising differentiating the GMP.
   91. The GMP of paragraphs 89-90, wherein the GMP is autologous with respect to a patient to be administered the cell.
   92. The GMP of paragraphs 89-90, wherein the GMP is allogenic with respect to a patient to be administered the cell.
   93. A myeloid progenitor cell transfected with a 5′-tiRNA of any one of paragraphs 1-9.
   94. The myeloid progenitor cell of paragraph 93, comprising differentiating the myeloid progenitor cell.
   95. The myeloid progenitor cell of paragraphs 93-94, wherein the myeloid progenitor cell is autologous with respect to a patient to be administered the cell.
   96. The myeloid progenitor cell of paragraphs 93-94, wherein the myeloid progenitor cell is allogenic with respect to a patient to be administered the cell.
   97. A method for modulating the differentiation of a stem-progenitor cell (SPC), comprising transfecting the SPC with a 5′-tiRNA of any one of paragraphs 1-9.
   98. The method of paragraph 97, wherein the stem-progenitor cells are induced pluripotent stem cells (iPSC).
   99. The method of paragraph 97, wherein the stem-progenitor are hematopoietic stem-progenitor cells (HSPC).
   100. The method of paragraph 97, wherein the stem-progenitor cells are myeloid progenitor cells.
   101. The method of paragraph 97, wherein the stem-progenitor cells are GMPs.
   102. The method of paragraph 97, wherein the stem-progenitor cells are isolated from a subject.
   103. The method of paragraph 97, wherein the stem-progenitor cells are peripheral blood stem-progenitor cells.

Other embodiments are within the claims.

### REFERENCES

Akashi, K., Traver, D., Miyamoto, T., and Weissman, I. L. (2000). A clonogenic common myeloid progenitor that gives rise to all myeloid lineages. Nature 404, 193-197.
Anders, S., Pyl, P. T., and Huber, W. (2015). HTSeq—a Python framework to work with high-throughput sequencing data. Bioinformatics 31, 166-169.
Anderson, P., and Ivanov, P. (2014). tRNA fragments in human health and disease. FEBS Lett 588, 4297-4304.
Avni, D., Shama, S., Loreni, F., and Meyuhas, O. (1994). Vertebrate mRNAs with a 5′-terminal pyrimidine tract are candidates for translational repression in quiescent cells: characterization of the translational cis-regulatory element. Mol Cell Biol 14, 3822-3833.
Baglio, S. R., Rooijers, K., Koppers-Lalic, D., Verweij, F. J., Perez Lanzon, M., Zini, N., Naaijkens, B., Perut, F., Niessen, H. W., Baldini, N., et al. (2015). Human bone marrow- and adipose-mesenchymal stem cells secrete exosomes enriched in distinctive miRNA and tRNA species. Stem Cell Res Ther 6, 127.
Bilic-Curcic, I., Kronenberg, M., Jiang, X., Bellizzi, J., Mina, M., Marijanovic, I., Gardiner, E. M., and Rowe, D. W. (2005). Visualizing levels of osteoblast differentiation by a two-color promoter-GFP strategy: Type I collagen-GFPcyan and osteocalcin-GFPtpz. Genesis 43, 87-98.
Blanco, S., Bandiera, R., Popis, M., Hussain, S., Lombard, P., Aleksic, J., Sajini, A., Tanna, H., Cortes-Garrido, R., Gkatza, N., et al. (2016). Stem cell function and stress response are controlled by protein synthesis. Nature 534, 335-340.
Bolger, A. M., Lohse, M., and Usadel, B. (2014). Trimmomatic: a flexible trimmer for Illumina sequence data. Bioinformatics 30, 2114-2120.
Borek, E., Baliga, B. S., Gehrke, C. W., Kuo, C. W., Belman, S., Troll, W., and Waalkes, T. P. (1977). High turnover rate of transfer RNA in tumor tissue. Cancer Res 37, 3362-3366.
Calvi, L. M., Sims, N. A., Hunzelman, J. L., Knight, M. C., Giovannetti, A., Saxton, J. M., Kronenberg, H. M., Baron, R., and Schipani, E. (2001). Activated parathyroid hormone/parathyroid hormone-related protein receptor in osteoblastic cells differentially affects cortical and trabecular bone. J Clin Invest 107, 277-286.
Calvo, K. R., Sykes, D. B., Pasillas, M., and Kamps, M. P. (2000). Hoxa9 immortalizes a granulocyte-macrophage colony-stimulating factor-dependent promyelocyte capable of biphenotypic differentiation to neutrophils or macrophages, independent of enforced meis expression. Mol Cell Biol 20, 3274-3285.
Chan, P. P., and Lowe, T. M. (2016). GtRNAdb 2.0: an expanded database of transfer RNA genes identified in complete and draft genomes. Nucleic Acids Res 44, D184-189.
Chen, G., Huang, A. C., Zhang, W., Zhang, G., Wu, M., Xu, W., Yu, Z., Yang, J., Wang, B., Sun, H., et al. (2018). Exosomal PD-L1 contributes to immunosuppression and is associated with anti-PD-1 response. Nature 560, 382-386.
Chiou, N. T., Kageyama, R., and Ansel, K. M. (2018). Selective Export into Extracellular Vesicles and Function of tRNA Fragments during T Cell Activation. Cell Rep 25, 3356-3370 e3354.
Conlan, R. S., Pisano, S., Oliveira, M. I., Ferrari, M., and Mendes Pinto, I. (2017). Exosomes as Reconfigurable Therapeutic Systems. Trends Mol Med 23, 636-650.
Crewe, C., Joffin, N., Rutkowski, J. M., Kim, M., Zhang, F., Towler, D. A., Gordillo, R., and Scherer, P. E. (2018). An Endothelial-to-Adipocyte Extracellular Vesicle Axis Governed by Metabolic State. Cell 175, 695-708 e613.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Dong, L., Yu, W. M., Zheng, H., Loh, M. L., Bunting, S. T., Pauly, M., Huang, G., Zhou, M., Broxmeyer, H. E., Scadden, D. T., et al. (2016). Leukaemogenic effects of Ptpn11 activating mutations in the stem cell microenvironment. Nature 539, 304-308.
Fricker, R., Brogli, R., Luidalepp, H., Wyss, L., Fasnacht, M., Joss, O., Zywicki, M., Helm, M., Schneider, A., Cristodero, M., et al. (2019). A tRNA half modulates translation as stress response in Trypanosoma brucei. Nat Commun 10, 118.
Fu, L., Niu, B., Zhu, Z., Wu, S., and Li, W. (2012). CD-HIT: accelerated for clustering the next-generation sequencing data. Bioinformatics 28, 3150-3152.
Goloviznina, N. A., Verghese, S. C., Yoon, Y. M., Taratula, O., Marks, D. L., and Kurre, P. (2016). Mesenchymal Stromal Cell-derived Extracellular Vesicles Promote Myeloid-biased Multipotent Hematopoietic Progenitor Expansion via Toll-Like Receptor Engagement. J Biol Chem 291, 24607-24617.
Goncalves, K. A., Silberstein, L., Li, S., Severe, N., Hu, M. G., Yang, H., Scadden, D. T., and Hu, G. F. (2016). Angiogenin Promotes Hematopoietic Regeneration by Dichotomously Regulating Quiescence of Stem and Progenitor Cells. Cell 166, 894-906.
Guzzi, N., Ciesla, M., Ngoc, P. C. T., Lang, S., Arora, S., Dimitriou, M., Pimkova, K., Sommarin, M. N. E., Munita, R., Lubas, M., et al. (2018). Pseudouridylation of tRNA-Derived Fragments Steers Translational Control in Stem Cells. Cell 173, 1204-1216 e1226.
Han, K., Jaimovich, A., Dey, G., Ruggero, D., Meyuhas, O., Sonenberg, N., and Meyer, T. (2014). Parallel measurement of dynamic changes in translation rates in single cells. Nat Methods 11, 86-93.
He, C., Bozler, J., Janssen, K. A., Wilusz, J. E., Garcia, B. A., Schorn, A. J., and Bonasio, R. (2021). TET2 chemically modifies tRNAs and regulates tRNA fragment levels. Nat Struct Mol Biol 28, 62-70.
Ivanov, P., Emara, M. M., Villen, J., Gygi, S. P., and Anderson, P. (2011). Angiogenin-induced tRNA fragments inhibit translation initiation. Mol Cell 43, 613-623.
Jeppesen, D. K., Fenix, A. M., Franklin, J. L., Higginbotham, J. N., Zhang, Q., Zimmerman, L. J., Liebler, D. C., Ping, J., Liu, Q., Evans, R., et al. (2019). Reassessment of Exosome Composition. Cell 177, 428-445 e418.
Johnstone, R. M., Adam, M., Hammond, J. R., Orr, L., and Turbide, C. (1987). Vesicle formation during reticulocyte maturation. Association of plasma membrane activities with released vesicles (exosomes). J Biol Chem 262, 9412-9420.
Jones, D. L., and Wagers, A. J. (2008). No place like home: anatomy and function of the stem cell niche. Nat Rev Mol Cell Biol 9, 11-21.
Kalajzic, I., Kalajzic, Z., Hurley, M. M., Lichtler, A. C., and Rowe, D. W. (2003). Stage specific inhibition of osteoblast lineage differentiation by FGF2 and noggin. J Cell Biochem 88, 1168-1176.
Keerthikumar, S., Chisanga, D., Ariyaratne, D., Al Saffar, H., Anand, S., Zhao, K., Samuel, M., Pathan, M., Jois, M., Chilamkurti, N., et al. (2016). ExoCarta: A Web-Based Compendium of Exosomal Cargo. J Mol Biol 428, 688-692.
Kfoury, Y., and Scadden, D. T. (2015). Mesenchymal cell contributions to the stem cell niche. Cell Stem Cell 16, 239-253.
Kim, H. K., Fuchs, G., Wang, S., Wei, W., Zhang, Y., Park, H., Roy-Chaudhuri, B., Li, P., Xu, J., Chu, K., et al. (2017). A transfer-RNA-derived small RNA regulates ribosome biogenesis. Nature 552, 57-62.
Kode, A., Manavalan, J. S., Mosialou, I., Bhagat, G., Rathinam, C. V., Luo, N., Khiabanian, H., Lee, A., Murty, V. V., Friedman, R., et al. (2014). Leukaemogenesis induced by an activating beta-catenin mutation in osteoblasts. Nature 506, 240-244.
Lenzo, J. C., O'Brien-Simpson, N. M., Cecil, J., Holden, J. A., and Reynolds, E. C. (2016). Determination of Active Phagocytosis of Unopsonized Porphyromonas gingivalis by Macrophages and Neutrophils Using the pH-Sensitive Fluorescent Dye pHrodo. Infect Immun 84, 1753-1760.
Li, H., and Durbin, R. (2009). Fast and accurate short read alignment with Burrows-Wheeler transform. Bioinformatics 25, 1754-1760.
Liu, J., Xu, Y., Stoleru, D., and Salic, A. (2012). Imaging protein synthesis in cells and tissues with an alkyne analog of puromycin. Proc Natl Acad Sci USA 109, 413-418.
Magee, R., and Rigoutsos, I. (2020). On the expanding roles of tRNA fragments in modulating cell behavior. Nucleic Acids Res 48, 9433-9448.
Mathieu, M., Martin-Jaular, L., Lavieu, G., and Thery, C. (2019). Specificities of secretion and uptake of exosomes and other extracellular vesicles for cell-to-cell communication. Nat Cell Biol 21, 9-17.
Melo, S. A., Luecke, L. B., Kahlert, C., Fernandez, A. F., Gammon, S. T., Kaye, J., LeBleu, V. S., Mittendorf, E. A., Weitz, J., Rahbari, N., et al. (2015). Glypican-1 identifies cancer exosomes and detects early pancreatic cancer. Nature 523, 177-182.
Mignone, J. L., Kukekov, V., Chiang, A. S., Steindler, D., and Enikolopov, G. (2004). Neural stem and progenitor cells in nestin-GFP transgenic mice. J Comp Neurol 469, 311-324.
Morhayim, J., van de Peppel, J., Braakman, E., Rombouts, E. W., Ter Borg, M. N., Dudakovic, A., Chiba, H., van der Eerden, B. C., Raaijmakers, M. H., van Wijnen, A. J., et al. (2016). Osteoblasts secrete miRNA-containing extracellular vesicles that enhance expansion of human umbilical cord blood cells. Sci Rep 6, 32034.
Morrison, S. J., and Scadden, D. T. (2014). The bone marrow niche for haematopoietic stem cells. Nature 505, 327-334.
Nielsen, M. S., Axelsen, L. N., Sorgen, P. L., Verma, V., Delmar, M., and Holstein-Rathlou, N. H. (2012). Gap junctions. Compr Physiol 2, 1981-2035.
Panaroni, C., Fulzele, K., Saini, V., Chubb, R., Pajevic, P. D., and Wu, J. Y. (2015). PTH Signaling in Osteoprogenitors Is Essential for B-Lymphocyte Differentiation and Mobilization. J Bone Miner Res 30, 2273-2286.
Patro, R., Duggal, G., Love, M. I., Irizarry, R. A., and Kingsford, C. (2017). Salmon provides fast and bias-aware quantification of transcript expression. Nat Methods 14, 417-419.
Raaijmakers, M. H., Mukherjee, S., Guo, S., Zhang, S., Kobayashi, T., Schoonmaker, J. A., Ebert, B. L., Al-Shahrour, F., Hasserjian, R. P., Scadden, E. O., et al. (2010). Bone progenitor dysfunction induces myelodysplasia and secondary leukaemia. Nature 464, 852-857.
Robinson, M. D., McCarthy, D. J., and Smyth, G. K. (2010). edgeR: a Bioconductor package for differential expression analysis of digital gene expression data. Bioinformatics 26, 139-140.
Rodda, S. J., and McMahon, A. P. (2006). Distinct roles for Hedgehog and canonical Wnt signaling in specification, differentiation and maintenance of osteoblast progenitors. Development 133, 3231-3244.
Sai Lakshmi, S., and Agrawal, S. (2008). piRNABank: a web resource on classified and clustered Piwi-interacting RNAs. Nucleic Acids Res 36, D173-177.
Schaefer, M., Pollex, T., Hanna, K., Tuorto, F., Meusburger, M., Helm, M., and Lyko, F. (2010). RNA methylation by Dnmt2 protects transfer RNAs against stress-induced cleavage. Genes Dev 24, 1590-1595.
Shurtleff, M. J., Yao, J., Qin, Y., Nottingham, R. M., Temoche-Diaz, M. M., Schekman, R., and Lambowitz, A. M. (2017). Broad role for YBX1 in defining the small noncoding RNA composition of exosomes. Proc Natl Acad Sci USA 114, E8987-E8995.
Silberstein, L., Goncalves, K. A., Kharchenko, P. V., Turcotte, R., Kfoury, Y., Mercier, F., Baryawno, N., Severe, N., Bachand, J., Spencer, J. A., et al. (2016). Proximity-Based Differential Single-Cell Analysis of the Niche to Identify Stem/Progenitor Cell Regulators. Cell Stem Cell 19, 530-543.
Silva, B. C., Costa, A. G., Cusano, N. E., Kousteni, S., and Bilezikian, J. P. (2011). Catabolic and anabolic actions of parathyroid hormone on the skeleton. J Endocrinol Invest 34, 801-810.
Speer, J., Gehrke, C. W., Kuo, K. C., Waalkes, T. P., and Borek, E. (1979). tRNA breakdown products as markers for cancer. Cancer 44, 2120-2123.
Stranford, D. M., and Leonard, J. N. (2017). Delivery of Biomolecules via Extracellular Vesicles: A Budding Therapeutic Strategy. Adv Genet 98, 155-175.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Sykes, D. B., Kfoury, Y. S., Mercier, F. E., Wawer, M. J., Law, J. M., Haynes, M. K., Lewis, T. A., Schajnovitz, A., Jain, E., Lee, D., et al. (2016). Inhibition of Dihydroorotate Dehydrogenase Overcomes Differentiation Blockade in Acute Myeloid Leukemia. Cell 167, 171-186 e115.
Tkach, M., and Thery, C. (2016). Communication by Extracellular Vesicles: Where We Are and Where We Need to Go. Cell 164, 1226-1232.
Tuorto, F., Liebers, R., Musch, T., Schaefer, M., Hofmann, S., Kellner, S., Frye, M., Helm, M., Stoecklin, G., and Lyko, F. (2012). RNA cytosine methylation by Dnmt2 and NSun2 promotes tRNA stability and protein synthesis. Nat Struct Mol Biol 19, 900-905.
Valadi, H., Ekstrom, K., Bossios, A., Sjostrand, M., Lee, J. J., and Lotvall, J. O. (2007). Exosome-mediated transfer of mRNAs and microRNAs is a novel mechanism of genetic exchange between cells. Nat Cell Biol 9, 654-659.
van Niel, G., D'Angelo, G., and Raposo, G. (2018). Shedding light on the cell biology of extracellular vesicles. Nat Rev Mol Cell Biol 19, 213-228.
Venezia, T. A., Merchant, A. A., Ramos, C. A., Whitehouse, N. L., Young, A. S., Shaw, C. A., and Goodell, M. A. (2004). Molecular signatures of proliferation and quiescence in hematopoietic stem cells. PLoS Biol 2, e301.
Wang, L., Mo, Q., and Wang, J. (2015). MlrExpress: A Database for Gene Coexpression Correlation in Immune Cells Based on Mutual Information and Pearson Correlation. J Immunol Res 2015, 140819.
Wei, Z., Batagov, A. O., Schinelli, S., Wang, J., Wang, Y., El Fatimy, R., Rabinovsky, R., Balaj, L., Chen, C. C., Hochberg, F., et al. (2017). Coding and noncoding landscape of extracellular RNA released by human glioma stem cells. Nat Commun 8, 1145.
Wen, D., Peng, Y., Liu, D., Weizmann, Y., and Mahato, R. I. (2016a). Mesenchymal stem cell and derived exosome as small RNA carrier and Immunomodulator to improve islet transplantation. J Control Release 238, 166-175.
Wen, S., Dooner, M., Cheng, Y., Papa, E., Del Tatto, M., Pereira, M., Deng, Y., Goldberg, L., Aliotta, J., Chatterjee, D., et al. (2016b). Mesenchymal stromal cell-derived extracellular vesicles rescue radiation damage to murine marrow hematopoietic cells. Leukemia 30, 2221-2231.
Yamasaki, S., Ivanov, P., Hu, G. F., and Anderson, P. (2009). Angiogenin cleaves tRNA and promotes stress-induced translational repression. J Cell Biol 185, 35-42.
Ying, W., Riopel, M., Bandyopadhyay, G., Dong, Y., Birmingham, A., Seo, J. B., Ofrecio, J. M., Wollam, J., Hernandez-Carretero, A., Fu, W., et al. (2017). Adipose Tissue Macrophage-Derived Exosomal miRNAs Can Modulate In Vivo and In Vitro Insulin Sensitivity. Cell 171, 372-384 e312.
Zerbino, D. R., Achuthan, P., Akanni, W., Amode, M. R., Barrell, D., Bhai, J., Billis, K., Cummins, C., Gall, A., Giron, C. G., et al. (2018). Ensembl 2018. Nucleic Acids Res 46, D754-D761.
Zhang, M., Xuan, S., Bouxsein, M. L., von Stechow, D., Akeno, N., Faugere, M. C., Malluche, H., Zhao, G., Rosen, C. J., Efstratiadis, A., et al. (2002). Osteoblast-specific knockout of the insulin-like growth factor (IGF) receptor gene reveals an essential role of IGF signaling in bone matrix mineralization. J Biol Chem 277, 44005-44012.

What is claimed is:

1. A method of treating a disease or disorder in a subject, the method comprising administering a therapeutically effective amount of a 5′-tiRNA to treat the disease or disorder in the subject.

2. The method of claim 1, wherein the disease or disorder is an infection.

3. The method of claim 2, wherein the infection is a fungal or bacterial infection.

4. The method of claim 3, wherein the fungus is Candida.

5. The method of claim 2, wherein the infection is a deep tissue infection.

6. The method of claim 1, wherein the disease or disorder is sepsis.

7. The method of claim 1, wherein the 5′-tiRNA increases the number of neutrophils, granulocytes or macrophages in the subject to treat the disease or disorder.

8. The method of claim 1, wherein the 5′-tiRNA increases myeloid cell production in the subject to treat the disease or disorder.

9. The method of claim 1, wherein the 5′-tiRNA is post-surgically administered to treat the disease or disorder.

10. The method of claim 1, wherein the 5′-tiRNA is administered to treat a trauma.

11. The method of claim 1, wherein the 5′-tiRNA increases reconstitution or recovery after a stem cell transplant, after radiation therapy, or after a chemical injury to bone marrow.

12. The method of claim 11, wherein the transplant is autologous.

13. The method of claim 11, wherein the transplant is allogenic.

14. The method of claim 1, wherein the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

15. The method of claim 14, wherein the 5′-tiRNA is intravenously administered.

16. The method of claim 14, wherein the 5′-tiRNA is formulated in a liposome, an exosome, or a lipid nanoparticle.

17. The method of claim 16, wherein the liposome, exosome, or lipid nanoparticle is intravenously administered.

18. The method of claim 14, wherein the 5′-tiRNA is present in a cell which is administered to treat a disease or disorder in the subject.

19. The method of claim 18, wherein the cell is an induced pluripotent stem cells (iPSC)-derived hematopoietic stem and progenitor cells (HSPC), a HSPC, a myeloid progenitor cell, a lymphoid progenitor cell, or a granulocyte-macrophage progenitor (GMP).

20. A method of delivering a 5′-tiRNA to a hematopoietic stem and/or progenitor cell (HSPC), the method comprising:
a.) transfecting the HSPC with a 5′-tiRNA in vitro; and
b.) optionally, culturing the HSPC in vitro;
thereby delivering the 5′-tiRNA to the HSPC.

21. The method of claim 20, wherein the HSPC is an iPSC-derived HSPC, an HSPC from a subject, a myeloid progenitor cell, a lymphoid progenitor cell, or a GMP.

22. The method of claim 20, wherein the HSPC is a human cell or sample.

23. The method of claim 20, wherein the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

24. An HSPC transfected with a 5′-tiRNA.

25. The HSPC of claim 24, wherein the 5′-tiRNA is 5′-ti-Pro-CGG-1-1: GGCUCGUUGGUCUAGGGGUAUGAUUCUCGCUUCG (SEQ ID NO: 1) or 5′-ti-Cys-GCA-10-1: GGGGGUAUAGCUCAGGGGUAGAGCAUUUGACUG (SEQ ID NO: 2) or both.

26. The HSPC of claim 24 or 25, wherein the HSPC is autologous with respect to a patient to be administered the cell.

27. The HSPC of claim 24 or 25, wherein the HSPC is allogenic with respect to a patient to be administered the cell.

28. An HSPC produced according to the method of claim 20.

29. The HSPC of claim 28, wherein the HSPC is an iPSC-derived HSPC, an HSPC from a subject, a myeloid progenitor cell, a lymphoid progenitor cell, or a GMP.
